(19) United States
(12) Patent Application Publication
(10) Pub. No.: US 2018/0186769 A1
(43) Pub. Date: Jul. 5, 2018

(54) 6-AMINO-5-FLUORO-5-(FLUOROMETHYL)-2,3,4,5-TETRAHYDROPYRIDIN-2-YL-PHENYL-5-(METHOXY-D3)-PYRAZINE-2-CARBOXAMIDES AND FLUORINATED DERIVATIVES THEREOF AS BACE1 INHIBITORS

(71) Applicant: H. Lundbeck A/S, Valby (DK)

(72) Inventors: Karsten Juhl, Valby (DK); Lena Tagmose, Valby (DK); Mauro Marigo, Valby (DK)

(21) Appl. No.: 15/848,013

(22) Filed: Dec. 20, 2017

(30) Foreign Application Priority Data
Dec. 21, 2016 (DK) PA201600778
Feb. 7, 2017 (DK) PA201700075

(51) Int. Cl. C07D 401/12; A61P 25/28

(52) U.S. Cl. CPC C07D 401/12; A61P 25/28

(57) ABSTRACT

The invention provides amidine compounds of Formula I

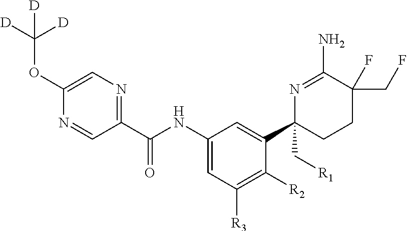

for the treatment of neurodegenerative or cognitive diseases, including Alzheimer's Disease.

### CROSS-REFERENCE TO RELATED APPLICATIONS

This application claims priority to Danish Application No. PA201600778, filed Dec. 21, 2016, and Danish Application No. PA201700075, filed Feb. 7, 2017, the entire contents of the aforementioned applications are hereby incorporated herein by reference.

### FIELD OF THE INVENTION

Beta-secretase (BACE) inhibitors are provided by the present invention. The use of the compounds to treat neurodegenerative and cognitive disorders is anticipated.

### BACKGROUND ART

Dementia is a clinical syndrome characterized by deficits in multiple areas of cognition that cannot be explained by normal aging, a noticeable decline in function, and an absence of delirium. In addition, neuropsychiatric symptoms and focal neurological findings are usually present. Dementia is further classified based on etiology. Alzheimer's disease (AD) is the most common cause of dementia, followed by mixed AD and vascular dementia, Lewy body dementia (DLB), and fronto-temporal dementia.

Beta-amyloid deposits and neurofibrillary tangles are considered to be major pathologic characterizations associated with AD which is characterized by the loss of memory, cognition, reasoning, judgment, and orientation. Also affected, as the disease progresses, are motor, sensory and linguistic abilities until global impairment of multiple cognitive functions occurs. Beta-amyloid deposits are predominantly an aggregate of the Abeta peptide, which in turn is a product of the proteolysis of amyloid precursor protein (APP) as part of the beta-amyloidogenic pathway. Abeta peptide results from the cleavage of APP at the C-terminals by one or more γ-secretases and at the N-terminus by beta-secretase 1 (BACE1) also known as aspartyl protease 2. BACE1 activity is correlated directly to the generation of Abeta peptide from APP.

Studies indicate that the inhibition of BACE1 impedes the production of Abeta peptide. Further, BACE1 co-localizes with its substrate APP in Golgi and endocytic compartments (Willem M, et al. Semin. Cell Dev. Biol, 2009, 20, 175-182). Knock-out studies in mice have demonstrated the absence of amyloid peptide formation while the animals are healthy and fertile (Ohno M, et al. Neurobiol. Dis., 2007, 26, 134-145). Genetic ablation of BACE1 in APP-overexpressing mice has demonstrated absence of plaque formation, and the reverse of cognitive deficits (Ohno M, et al. Neuron; 2004, 41, 27-33). BACE1 levels are elevated in the brains of sporadic AD patients (Hampel and Shen, Scand. J. Clin. Lab. Invest. 2009, 69, 8-12).

These convergent findings indicate that the inhibition of BACE1 may be a therapeutic target for the treatment of AD as well as neurodegenerative or cognitive disorders for which the reduction of Abeta deposits is beneficial.

AstraZeneca announced the discovery of AZD3839, a potent BACE1 inhibitor clinical candidate for the treatment of AD (Jeppsson, F., et al. J. Biol. Chem., 2012, 287, 41245-41257) in October 2012. The effort which led to the discovery of AZD3839 was further described in Ginman, T., et al. J. Med. Chem., 2013, 56, 4181-4205. The Ginman publication describes the issues which were overcome in connection with the discovery and identification of AZD3839. These issues related to poor blood brain barrier penetration and P-glycoprotein mediated efflux of the compounds resulting in low brain exposure.

The Ginman manuscript hypothesized that the differences in brain exposure would largely be due to the core structures and Structure Activity Relationship data was provided wherein the in vitro properties on the reported compounds were given in four tables according to core sub-types. In table 6, a series of amidine containing compounds are described that were considered interesting from an activity perspective. However, the data suggested that the amidine containing core did not exhibit a favourable blood brain barrier permeability profile.

WO2015/124576 discloses tri-fluorinated amidines as BACE inhibitors. WO2016/075063 discloses tetrafluorinated amidines as BACE inhibitors.

Researchers from Hoffmann-La Roche and Siena Biotech also reported the discovery of amidine containing compounds (Woltering, T. J., et al. Bioorg. Med. Chem. Lett. 2013, 23, 4239-4243). These compounds (compounds 17 and 18 in the paper) were found not to have any in vivo effect (lack of Abeta40 reduction in brain in wild type mice).

Contrary to the teachings of Ginman, et al. and Woltering, T. J., et al., the present inventors have discovered a series of amidine compounds which are brain penetrating. Accordingly, the present invention relates to novel compounds having BACE1 inhibitory activity, to their preparation, to their medical use and to medicaments comprising them.

### SUMMARY OF THE INVENTION

An objective of the present invention is to provide compounds that substantially inhibit BACE1. Accordingly, the present invention relates to compounds of Formula I:

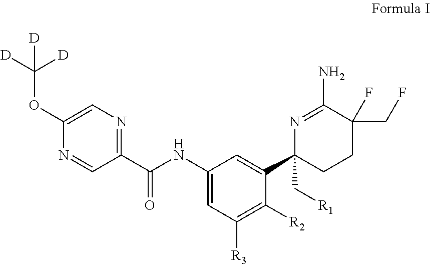

wherein R₁ is hydrogen or a fluorine;
R₂ is hydrogen or a halogen;
R₃ is hydrogen or a halogen;
D is deuterium;
and pharmaceutically acceptable salts thereof.

In one embodiment, the invention provides compounds of Formula I or pharmaceutically acceptable salts thereof for use in therapy.

The invention further provides a pharmaceutical composition comprising a compound of Formula I or a pharmaceutically acceptable salt thereof and a pharmaceutically acceptable carrier.

In one embodiment, the invention provides the use of a compound of Formula I or a pharmaceutically acceptable salt thereof in the manufacture of a medicament for the treatment of neurodegenerative or cognitive disorder.

In one embodiment, the invention provides a compound of Formula I or a pharmaceutically acceptable salt thereof for use in a method for the treatment of a neurodegenerative or cognitive disorder.

The present invention provides a method of treating a neurodegenerative or cognitive disorder comprising administering a therapeutically effective amount of a compound of Formula I or a pharmaceutically acceptable salt thereof to a patient in need thereof.

### DETAILED DESCRIPTION OF THE INVENTION

Amidine compounds of Formula I

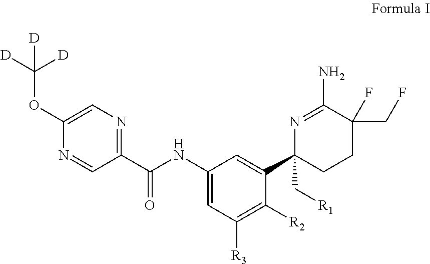

were surprisingly found to have favourable blood brain barrier permeability and affinity for BACE.

The term “halogen” is intended to mean fluorine, chlorine and bromine.

The phrase “therapeutically effective amount” when applied to a compound of the invention is intended to denote an amount of the compound that is sufficient to ameliorate, palliate, stabilize, reverse, slow or delay the progression of a disorder or disease state, or of a symptom of the disorder or disease. In an embodiment, the method of the present invention provides for administration of combinations of compounds. In such instances, the “therapeutically effective amount” is the amount of a compound of the present invention in the combination sufficient to cause the intended biological effect.

The term “treatment” or “treating” as used herein means ameliorating or reversing the progress or severity of a disease or disorder, or ameliorating or reversing one or more symptoms or side effects of such disease or disorder. “Treatment” or “treating”, as used herein, also means to inhibit or block, as in retard, arrest, restrain, impede or obstruct, the progress of a system, condition or state of a disease or disorder. For purposes of this invention, “treatment” or “treating” further means an approach for obtaining beneficial or desired clinical results, where “beneficial or desired clinical results” include, without limitation, alleviation of a symptom, diminishment of the extent of a disorder or disease, stabilized (i.e., not worsening) disease or disorder state, delay or slowing of a disease or disorder state, amelioration or palliation of a disease or disorder state, and remission of a disease or disorder, whether partial or total.

A first aspect of the invention is directed to a compound of Formula I

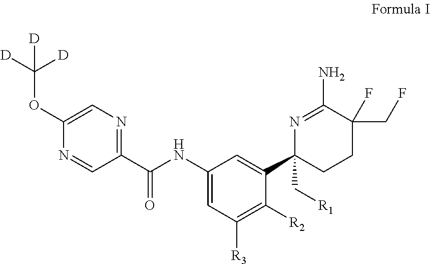

wherein R₁ is hydrogen or a fluorine; R₂ is hydrogen or a halogen; R₃ is hydrogen or a halogen; and D is deuterium;
and to pharmaceutically acceptable salts thereof.

Compounds of Formula I may be as a mixture of diastereomers or as one of the diastereomeric forms. Accordingly, compounds of Formula I may be of the Formula Ia or Ib

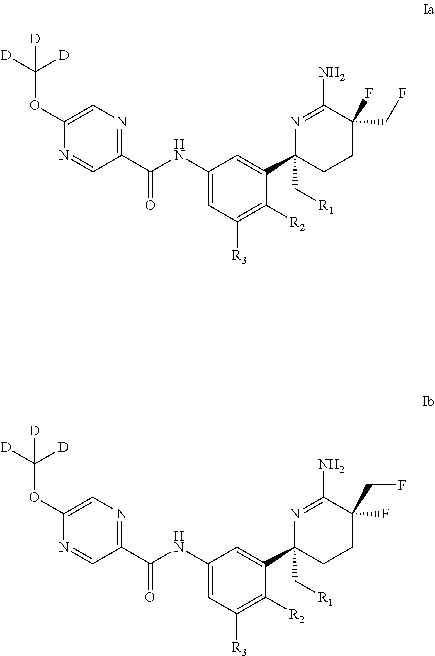

wherein R₁ is hydrogen or a fluorine; R₂ is hydrogen or a halogen; R₃ is hydrogen or a halogen; and D is deuterium.

In one embodiment, each of R₁, R₂ and R₃ is hydrogen. In an alternative embodiment, at least one of R₂ and R₃ is a halogen. In a further embodiment, at least one of R₁, R₂ and R₃ is fluorine. In an alternative embodiment, at least R₂ is a halogen, preferably wherein at least R₂ is fluorine.

In one embodiment, a compound of the present invention is selected from the group consisting of
N-(3-((2S,5S)-6-amino-5-fluoro-5-(fluoromethyl)-2-methyl-2,3,4,5-tetrahydropyridin-2-yl)-4-fluorophenyl)-5-(methoxy-d₃)pyrazine-2-carboxamide;
N-[3-[(2S,5R)-6-amino-5-fluoro-5-(fluoromethyl)-2-methyl-2,3,4,5-tetrahydropyridin-2-yl]-4,5-difluoro-phenyl]-5-(methoxy-d₃)pyrazine-2-carboxamide;
N-[3-[(2S,5R)-6-amino-5-fluoro-2,5-bis(fluoromethyl)-2,3,4,5-tetrahydropyridin-2-yl]-4-fluoro-phenyl]-5-(methoxy-d₃)pyrazine-2-carboxamide;
N-[3-[(2S,5S)-6-amino-5-fluoro-2,5-bis(fluoromethyl)-2,3,4,5-tetrahydropyridin-2-yl]-4-fluoro-phenyl]-5-(methoxy-d₃)pyrazine-2-carboxamide, and
N-L[3-[(2S,5S)-6-amino-5-fluoro-5-(fluoromethyl)-2-methyl-2,3,4,5-tetrahydropyridin-2-yl]-4,5-difluoro-phenyl]-5-(methoxy-d₃)pyrazine-2-carboxamide; or a pharmaceutically acceptable salt thereof.

It is recognized that elements are present in natural isotopic abundances in most synthetic compounds, and result in inherent incorporation of deuterium. The natural isotopic abundance of hydrogen isotopes such as deuterium is about 0.015%. Thus, as used herein, designation of an atom as deuterium at a position indicates that the abundance of deuterium is significantly greater than the natural abundance of deuterium. Any atom not designated as a particular isotope is intended to represent any stable isotope of that atom, as will be apparent to the ordinarily skilled artisan. Any atom not designated as deuterium is present at about its natural isotopic abundance. In some embodiments, designation of a position as “D” in a compound has a minimum deuterium incorporation of greater than about 50% at that position. In some embodiments, designation of a position as “D” in a compound has a minimum deuterium incorporation of greater than about 60% at that position. In some embodiments, designation of a position as “D” in a compound has a minimum deuterium incorporation of greater than about 65% at that position. In some embodiments, designation of a position as “D” in a compound has a minimum deuterium incorporation of greater than about 70% at that position. In some embodiments, designation of a position as “D” in a compound has a minimum deuterium incorporation of greater than about 75% at that position. In some embodiments, designation of a position as “D” in a compound has a minimum deuterium incorporation of greater than about 80% at that position. In some embodiments, designation of a position as “D” in a compound has a minimum deuterium incorporation of greater than about 85% at that position. In some embodiments, designation of a position as “D” in a compound has a minimum deuterium incorporation of greater than about 90% at that position. In some embodiments, designation of a position as “D” in a compound has a minimum deuterium incorporation of greater than about 95% at that position. In some embodiments, designation of a position as “D” in a compound has a minimum deuterium incorporation of greater than about 97% at that position. In some embodiments, designation of a position as “D” in a compound has a minimum deuterium incorporation of greater than about 99% at that position.

The present invention is based on the discovery that compounds of Formula I are inhibitors of BACE1, and as such, are useful for the treatment of disorders which pathological characteristics comprise beta-amyloid deposits and neurofibrillary tangles, such as neurodegenerative or cognitive disorders.

The compounds of the present invention are, as discussed above, for use in the treatment of Alzheimer's disease due to their effects on beta-amyloid deposits and neurofibrillary tangles. This includes familial Alzheimer's disease where patients carry mutations on specific genes intimately involved in the production of Abeta peptide. It is, however, important to note that aggregates of Abeta peptide is not limited to familial Alzheimer's disease but is similarly an important pathophysiological characteristics of the more common sporadic Alzheimer's disease [Mol Cell Neurosci, 66, 3-11, 2015]. Thus, the compounds of the present invention are also for use in the treatment of sporadic Alzheimer's disease.

As can be seen from Table 4, the inventors have discovered that the compounds of the invention have improved clearance compared to their non-deuterated counterparts (Compound Z) and compared to other deuterated compounds (Compound Y).

The compounds of the present invention are for use in the treatment of early-stage Alzheimer's disease, i.e. disease stages where the biological and structural changes have started but the clinical manifestations of the disease have not yet become evident or are not yet well developed. Early-stage Alzheimer's disease may, in fact, start years before any clinical manifestation of the disease becomes manifest. Early-stage Alzheimer's disease includes prodromal Alzheimer's disease, preclinical Alzheimer's disease and mild cognitive impairment. Although mild cognitive impairment may be unrelated to Alzheimer's disease it is often a transitional stage to Alzheimer's disease or due to Alzheimer's disease. Preclinical and prodromal Alzheimer's disease are asymptomatic stages, and they are typically diagnosed by the presence of Alzheimer's disease related biomarkers. In this context the compounds of the present invention are believed to be useful in slowing down the progression of early-stage Alzheimer's disease, such as mild cognitive impairment to Alzheimer's disease. The compounds of the present invention are also believed to be useful in the treatment of memory loss, attention deficits, and dementia associated with Alzheimer's disease.

Other diseases to which the present invention pertains, in addition to the continuum of Alzheimer's disease, are characterized by beta-amyloid deposits and neurofibrillary tangles. Accordingly, a further embodiment of the invention is directed to the treatment of a diseases characterized by beta-amyloid deposits and neurofibrillary tangles. This includes Trisomy 21 also known as Down's syndrome. Patients suffering from Down's syndrome have an extra chromosome 21 which chromosome contains the gene for the amyloid precursor protein (APP). The extra chromosome 21 leads to overexpression of APP, which leads to increased levels of Abeta peptide, which eventually causes the markedly increased risk of developing Alzheimer's disease seen in Down's syndrome patients [Alzheimer's & Dementia, 11, 700-709, 201]. Cerebral amyloid angiopathy is also characterized by beta-amyloid deposits and neurofibrillary tangles in blood vessels of the central nervous system [Pharmacol Reports, 67, 195-203, 2015] and is as such expected to be treatable with compounds of the present invention.

In one embodiment, the present invention provides a method of treating a disease selected from Alzheimer's disease (familial or sporadic), preclinical Alzheimer's disease, prodromal Alzheimer's disease, mild cognitive impairment, Down's syndrome and cerebral amyloid angiopathy, the method comprising the administration of a therapeutically effective amount of a compound of Formula I or a pharmaceutically acceptable salt thereof to a patient in need thereof.

The present invention further provides a method of inhibiting BACE1 in a patient comprising administering to a patient in need thereof a therapeutically effective amount of a compound of Formula I or a pharmaceutically acceptable salt thereof.

The present invention also provides a method of inhibiting beta-secretase mediated cleavage of amyloid precursor protein comprising administering to a patient in need of such treatment a therapeutically effective amount a compound of Formula I or a pharmaceutically acceptable salt thereof.

In further embodiments, the present invention provides the use of a compound of Formula I or a pharmaceutically acceptable salt thereof for the manufacture of a medicament for the treatment of disease selected from Alzheimer's disease (familial or sporadic), preclinical Alzheimer's disease, prodromal Alzheimer's disease, mild cognitive impairment, Down's syndrome or cerebral amyloid angiopathy.

The present invention also provides the use of a compound of Formula I or a pharmaceutically acceptable salt thereof for the manufacture of a medicament for the inhibition of BACE1. The present invention further provides the use of a compound of Formula I or a pharmaceutically acceptable salt thereof for the manufacture of a medicament for the inhibition of production or accumulation of Abeta peptide.

In one embodiment, the present invention provides a compound of Formula I or a pharmaceutically acceptable salt thereof for use in a method for the treatment of a disease selected form Alzheimer's disease (familial or sporadic), preclinical Alzheimer's disease, prodromal Alzheimer's disease, mild cognitive impairment, Down's syndrome or cerebral amyloid angiopathy.

In one embodiment, the present invention relates to a compound of Formula I or a pharmaceutically acceptable salt thereof for use in a method for inhibiting of BACE1 or in a method for inhibiting of production or accumulation of Abeta peptide.

The compounds of the present invention are as demonstrated in the examples potent inhibitors of BACE1 and capable of lowering the level of Abeta peptide in rat brain and plasma, and said compounds are thus believed to be useful in the treatment of neurodegenerative and cognitive disorders which pathological characteristics comprise Abeta deposits and neurofibrilary tangles, such as e.g. Alzheimer's disease. It may be beneficial to combine a compound of the present invention with another treatment paradigm useful in the treatment of such disease, e.g. Alzheimer's disease.

Accordingly, one embodiment of the invention is directed to a compound of Formula I, or a pharmaceutically acceptable salt thereof, administered in combination with a second pharmaceutical compound wherein said second pharmaceutical compound is effective, alone or in combination with the compound of Formula I or a pharmaceutically acceptable salt thereof, for use in a method for the treatment of a disease selected form Alzheimer's disease (familial or sporadic), preclinical Alzheimer's disease, prodromal Alzheimer's disease, mild cognitive impairment, Down's syndrome or cerebral amyloid angiopathy. The combination of the second pharmaceutical compound and the compound of Formula I or a pharmaceutically acceptable salt thereof may be a co-Formulation, separate Formulations administered simultaneously, or separate Formulations administered non-simultaneously as part of an overall treatment regime. One embodiment of the invention is directed to a pharmaceutical composition comprising a compound of Formula I, or a pharmaceutically acceptable salt thereof, and a second pharmaceutical compound, wherein said second pharmaceutical compound is effective, alone or in combination with the compound of Formula I or a pharmaceutically acceptable salt thereof, for use in a method for the treatment of a disease selected form Alzheimer's disease (familial or sporadic), preclinical Alzheimer's disease, prodromal Alzheimer's disease, mild cognitive impairment, Down's syndrome or cerebral amyloid angiopathy. A related but alternative aspect of the invention is directed to a method for the treatment of a disease selected from Alzheimer's disease (familial or sporadic), preclinical Alzheimer's disease, prodromal Alzheimer's disease, mild cognitive impairment, Down's syndrome and cerebral amyloid angiopathy comprising the administration of a combination of a compound of Formula I, or a pharmaceutically acceptable salt thereof, with a second pharmaceutical compound wherein said second pharmaceutical compound is effective, alone or in combination with the compound of Formula I or a pharmaceutically acceptable salt thereof, for use in a method for said treatment. The method of treatment may be such that the combination may be by means of a co-Formulation, by means of separate Formulations and simultaneous administration, or by means of separate Formulations and non-simultaneous administration as part of an overall treatment regime.

In one embodiment, a mammal is a human. In one embodiment, the patient is a human patient.

### Pharmaceutically Acceptable Salts

The compounds of this invention are generally used as the free base or as a pharmaceutically acceptable salt thereof. Pharmaceutically acceptable salts of a compound of Formula I are prepared e.g. in a conventional manner by treating a solution or suspension of a free base of Formula I with a molar equivalent of a pharmaceutically acceptable acid. Representative examples of suitable organic and inorganic acids are described below. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines (for example, 8-bromotheophylline and the like). Further examples of pharmaceutically acceptable inorganic or organic acid addition salts include the pharmaceutically acceptable salts listed in S. M. Berge, et al., J. Pharm. Sci., 1977, 66, 2.

Furthermore, the compounds of this invention may exist in unsolvated as well as in solvated forms with pharmaceutically acceptable solvents such as water, ethanol and the like.

The compounds of the present invention may have one or more asymmetric centres and it is intended that any optical isomers (i.e. enantiomers or diastereomers), as separated, pure or partially purified optical isomers and any mixtures thereof including racemic mixtures, i.e. a mixture of stereoisomeres, are included within the scope of the invention.

In this context is understood that when specifying the enantiomeric form, then the compound is in enantiomeric excess, e.g. essentially in a pure form. Accordingly, one embodiment of the invention relates to a compound of the invention having an enantiomeric excess of at least 60%, at least 70%, at least 80%, at least 85%, at least 90%, at least 96%, preferably at least 98%. Racemic forms may be resolved into the optical antipodes by known methods, for example, by separation of diastereomeric salts thereof with an optically active acid, and liberating the optically active amine compound by treatment with a base. Separation of such diastereomeric salts can be achieved, e.g. by fractional crystallization. The optically active acids suitable for this purpose may include, but are not limited to d- or l-tartaric, mandelic or camphorsulfonic acids. Another method for resolving racemates into the optical antipodes is based upon chromatography on an optically active matrix. The compounds of the present invention may also be resolved by the formation and chromatographic separation of diastereomeric derivatives from chiral derivatizing reagents, such as, chiral alkylating or acylating reagents, followed by cleavage of the chiral auxiliary. Any of the above methods may be applied either to resolve the optical antipodes of the compounds of the invention per se or to resolve the optical antipodes of synthetic intermediates, which can then be converted by methods described herein into the optically resolved final products which are the compounds of the invention.

Additional methods for the resolution of optical isomers, known to those skilled in the art, may be used. Such methods include those discussed by J. Jacques, A. Collet and S. Wilen in Enantiomers, Racemates, and Resolutions, John Wiley and Sons, New York, 1981. Optically active compounds can also be prepared from optically active starting materials.

### Pharmaceutical Compositions

The present invention further provides a pharmaceutical composition comprising a compound of Formula I or a pharmaceutically acceptable salt thereof and a pharmaceutically acceptable carrier. The present invention also provides a pharmaceutical composition comprising a specific compound disclosed in the Experimental Section or a pharmaceutically acceptable salt thereof and a pharmaceutically acceptable carrier.

The compounds of the invention may be administered alone or in combination with pharmaceutically acceptable carriers or excipients, in either single or multiple doses. The pharmaceutical compositions according to the invention may be Formulated with pharmaceutically acceptable carriers or diluents as well as any other known adjuvants and excipients in accordance with conventional techniques such as those disclosed in Remington: The Science and Practice of Pharmacy, 22th Edition, Gennaro, Ed., Mack Publishing Co., Easton, Pa., 2013. Pharmaceutical compositions for oral administration include solid dosage forms such as capsules, tablets, dragees, pills, lozenges, powders and granules. Where appropriate, the compositions may be prepared with coatings such as enteric coatings or they may be Formulated so as to provide controlled release of the active ingredient such as sustained or prolonged release according to methods well known in the art. Liquid dosage forms for oral administration include solutions, emulsions, suspensions, syrups and elixirs. Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Other suitable administration forms include, but are not limited to, suppositories, sprays, ointments, creams, gels, inhalants, dermal patches and implants.

Typical oral dosages range from about 0.01 to about 100 mg/kg body weight per day.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of Formula I or a pharmaceutically acceptable salt thereof and a pharmaceutically acceptable carrier are readily administered in a variety of dosage forms suitable for the disclosed routes of administration. The Formulations may conveniently be presented in unit dosage form by methods known in the art of pharmacy. If a solid carrier is used for oral administration, the preparation may be tabletted, placed in a hard gelatin capsule in powder or pellet form or it may be in the form of a troche or lozenge. The amount of solid carrier will vary widely but will range from about 25 mg to about 1 g per dosage unit. If a liquid carrier is used, the preparation may be in the form of a syrup, emulsion, soft gelatin capsule or sterile injectable liquid such as an aqueous or nonaqueous liquid suspension or solution.

### EXPERIMENTAL SECTION

The compounds of the present invention of general Formula I, wherein R¹, R² and R³ are as defined above can be prepared by the methods outlined in the following reaction schemes 1-5 and in the examples. In the described methods, it is possible to make use of variants or modifications, which are themselves known to chemists skilled in the art or could be apparent to the person of ordinary skill in this art. Furthermore, other methods for preparing compounds of the invention will be apparent to the person skilled in the art in light of the following reaction schemes and examples.

It may be necessary to incorporate protection and deprotection strategies for substituents such as amino, amido, keto and hydroxyl groups in the synthetic methods described below to synthesize the compounds of Formula I. Methods for protection and deprotection of such groups are well known in the art, and may be found in T. Greene, et al., Protective Groups in Organic Synthesis, 1991, 2^{nd} Edition, John Wiley & Sons, New York.

For compounds, which can exist as a mixture or equilibrium between two or more tautomers, only one tautomer is represented in the schemes, although it may not be the most stable tautomer. For compounds, which can exist in enantiomeric, stereoisomeric or geometric isomeric forms their geometric configuration is specified; otherwise the structure represents a mixture of stereoisomers.

Analytical LC-MS data was obtained using the following methods.

Method A: LC-MS was run on Waters Aquity UPLC with PDA detector (operating at 254 nm), ELS detector, and TQD MS-detector equipped with APPI-source operating in positive ion mode.
LC-conditions: The column was Acquity UPLC BEH C18 1.7 μm; 2.1×150 mm operating at 60° C. with 1.2 ml/min of a binary gradient consisting of water+0.05% trifluoroacetic acid (A) and acetonitrile+5% water+0.035% trifluoroacetic acid (B). Gradient: 0.00 min: 10% B; 1.00 min: 100% B; 1.01 min: 10% B; 1.15 min: 10% B. Total run time: 1.15 minutes. Method B: LC-MS was run on Waters Acquity UPLC-MS with a PDA detector (operating at 254 nm), ELS detector, and TQ-MS equipped with ESI-source operating in positive ion mode. LC-conditions: The column was XSelect CSH C18 3.5 μm; 4.6×50 mm operating at 25° C. with 2.5 ml/min of a binary gradient consisting of water+0.1% formic acid (A) and acetonitrile++0.1% formic acid (B). Gradient: 0.00 min: 3% B; 2.50 min: 90% B; 3.50 min: 90% B; 3.55 min: 3% B; 4 min: 3% B. Total run time: 4 minutes.

¹H NMR spectra were recorded at 600 MHz on a Bruker Avance AV-III-600 instrument or at 400 MHz on a Bruker Avance AV-III-400 instrument or a Varian 400 instrument. Chemical shift values are expressed in ppm-values relative. The following abbreviations are used for multiplicity of NMR signals: s=singlet, d=doublet, t=triplet, q=quartet, dd=double doublet, ddd=double double doublet, dt=double triplet, br=broad, and m=multiplet.

Compounds of general Formula XIII may be prepared as shown in Scheme 1.

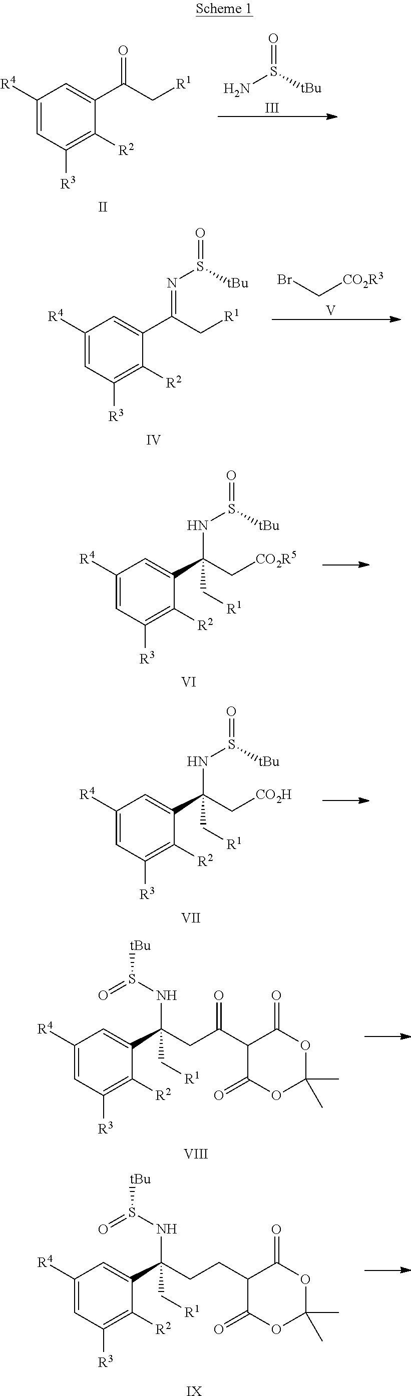

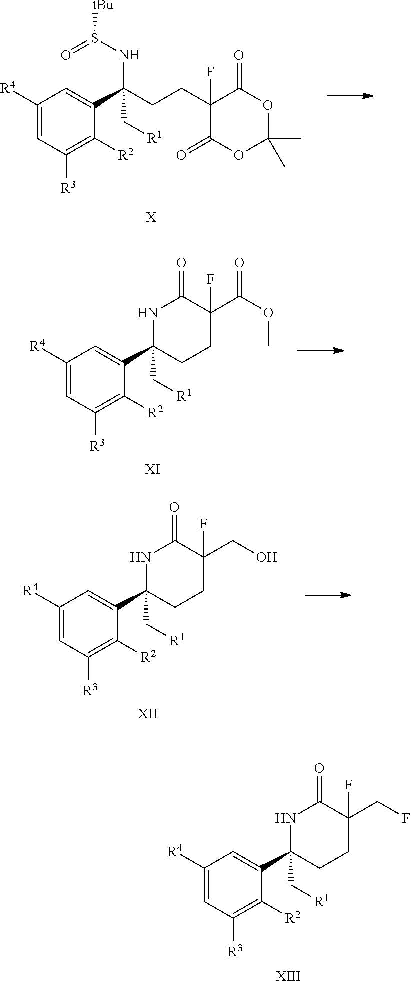

where R¹, R² and R³ are as defined for Formula I, R⁴ is hydrogen or a nitro group and R⁵ is an alkyl group such as methyl or ethyl.

Compounds of general Formula IV (Scheme 1) may be prepared by reacting compounds of Formula II with a sulfinamide such as III in the presence of a Lewis acid/drying agent such as titanium tetraethoxide. Treatment of compounds of general Formula IV with compounds of general Formula V such as ethyl bromoacetate in the presence of Zn powder or in the presence of diethyl zinc and tris(triphenylphosphine)rhodium(I) chloride gives compounds of general Formula VI (Hilpert, H. et al J. Med. Chem. 2013, 56, 3980-3995). Hydrolysis of compounds of general Formula VI with an aqueous base such as sodium hydroxide in water gives compounds of general Formula VII. Activation of the acid group of compounds of general Formula VII with a coupling reagent such as 1-ethyl-3-(3-dimethylaminopropyl)carbodiimide (EDC) in the presence of a catalyst such as 4-dimethylaminopyridine (DMAP) followed by reaction with Meldrum's acid (2,2-dimethyl-1,3-dioxane-4,6-dione) acid gives compounds of general Formula VIII. Reduction of compounds of general Formula VIII with a reducing reagent such as sodium borohydride in a solvent such as acetic acid gives compounds of general Formula IX. Compounds of general Formula IX can be fluorinated with a fluorination reagent such as N-fluorodibenzenesulfonamide (NFSi) in the presence of a base such as 1,8-diazabicyclo[5.4.0]undec-7-ene (DBU) to give compounds of general Formula X. Treating compounds of general Formula X with an acid such as hydrochloric acid in methanol will cleave the sulfinamide bond and transesterification will occur on the two ester moieties. Treatment of the resulting intermediate with a base such as potassium carbonate in methanol gives compounds of general Formula XI as a mixture of two diastereomers. The ester moiety of compounds of general Formula XI can be reduced with a reducing reagent such as sodium borohydride to give compounds of general Formula XII. The mixture of two diastereomers of compounds of general Formula XII can be converted to compounds of general Formula XIII as a mixture of two diastereomers by treatment with reagents such as nonafluorobutanesulfonyl fluoride (NfF) and a base such as triethylamine followed by treatment with a reagent such as tetra-N-butylammonium fluoride (TBAF). The mixture of two diastereomers of compounds of general Formula XIII when R⁴ is a nitro group can be separated by chromatography to give compounds of general Formulae XIVa and XIVb (Scheme 2).

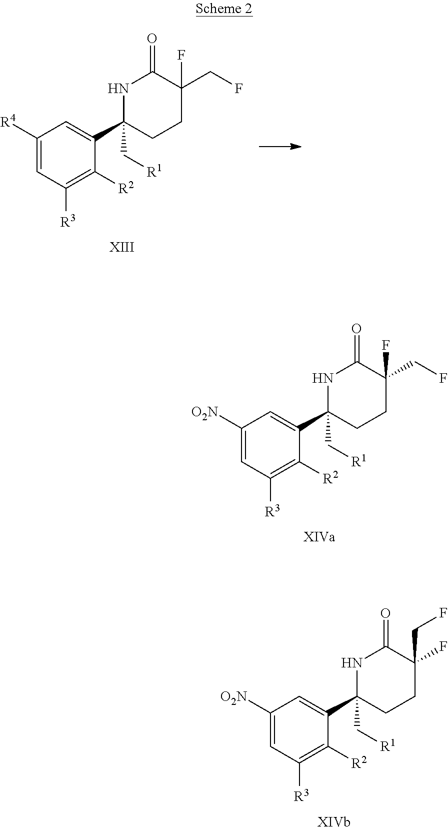

where R¹, R² and R³ are as defined under Formula I and R⁴ is a nitro group.

Compounds of general Formulae XIVa and XIVb may be prepared as shown in Scheme 3.

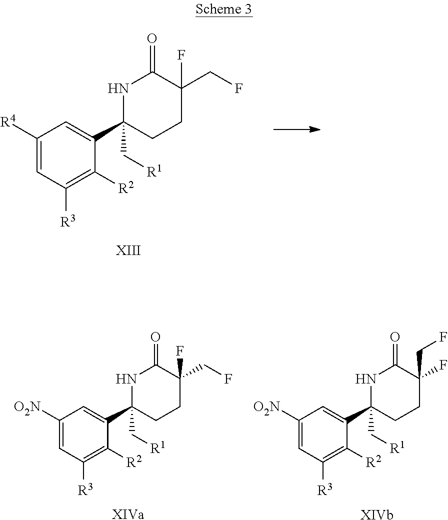

where R¹, R² and R³ are as defined under Formula I and R⁴ is hydrogen.

Treatment of a mixture of two diastereomers of compounds of general Formula XIII with nitric acid in sulfuric acid and trifluoroacetic acid gives compounds of general Formulae XIVa and XIVb which can be separated by chromatography (Scheme 3).

Compounds of general Formulae XVIa and XVIb may be prepared as shown in Scheme 4.

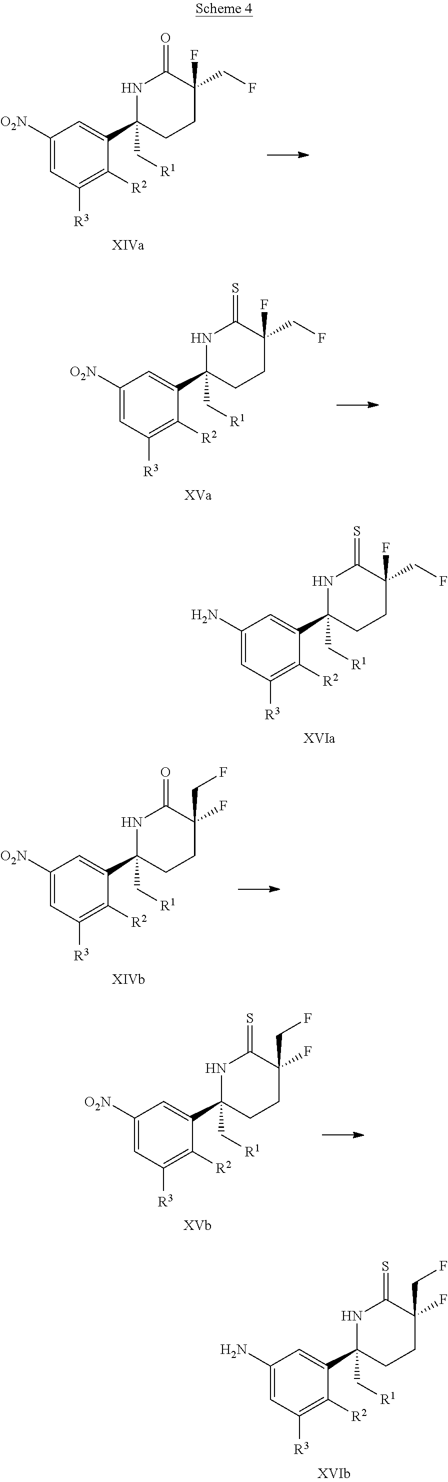

where R¹, R² and R³ are as defined under Formula I.

Treatment of compounds of general Formula XIVa (Scheme 4) with a reagent such as Lawesson's reagent (2,4-bis(4-methoxyphenyl)-1,3,2,4-dithiadiphosphetane-2,4-disulfide) gives compounds of general Formula XVa. Reduction of the nitro group of compounds of general Formula XVa gives compounds of general Formula XVIa. The same procedures can be used to prepare compounds of general Formula XVIb starting from compounds of general Formula XIVb. The same procedure can also be used used to prepare compounds of general Formulae XVIa and XVIb, by starting from a mixture of compounds of general Formulae XIVa and XIVb followed by separation of the resulting mixture of compounds of general Formulae XVIa and XVIb, e.g. by chromatography.

Compounds of general Formula I may be prepared as shown in Scheme 5.

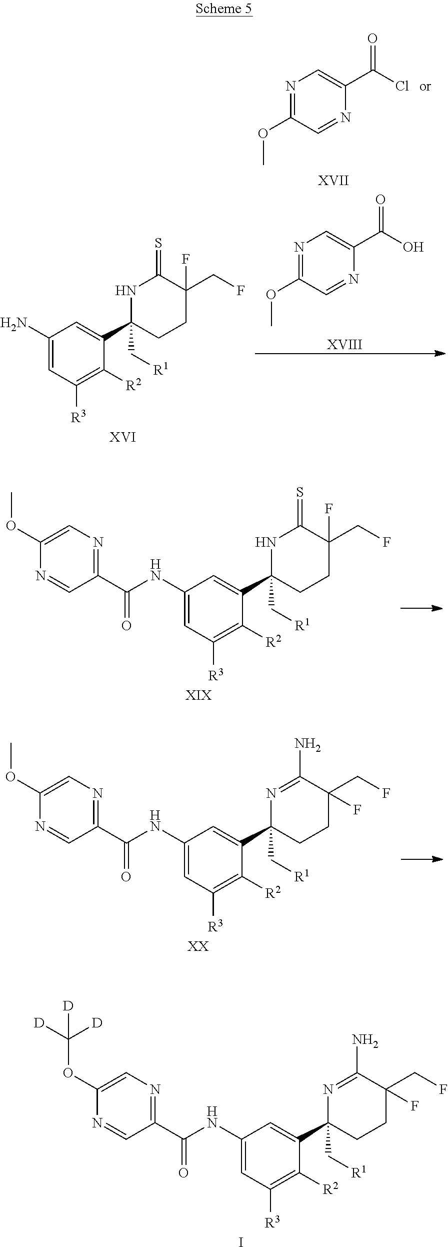

where R¹, R² and R³ are as defined under Formula I

Compounds of general Formula XIX (Scheme 5) may be prepared by reacting compounds of general Formula XVI with a carboxylic acid chloride of general Formula XVII or by reaction with a carboxylic acid of general Formula XVIII using procedures known to chemists skilled in the art. Treatment of compounds of general Formula XIX with ammonia gives compounds of general Formula XX. In some cases, the addition of an oxidizing reagent such as tert-butyl hydroperoxide might be necessary to facilitate the reaction. Treatment of compounds of general Formula XX with sodium trideuteriomethanolate in tetradeuteriomethonol gives compounds of general Formula I.

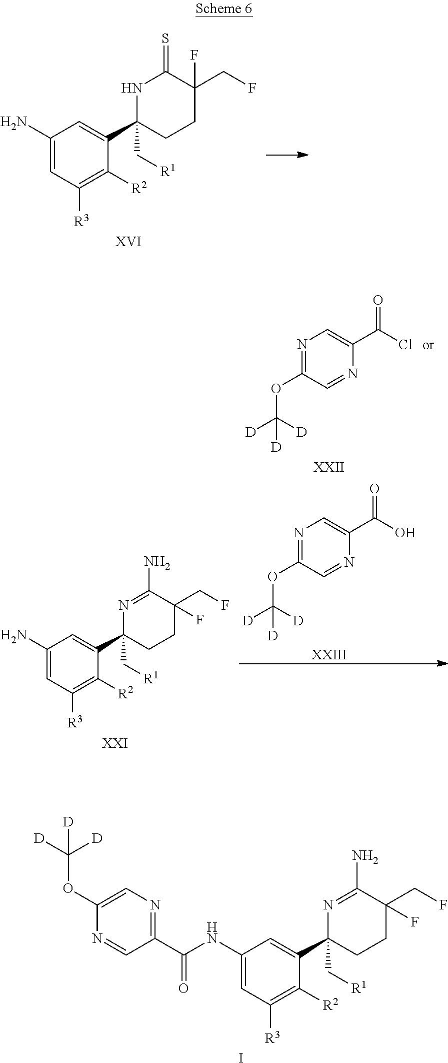

where R¹, R² and R³ are as defined under Formula I.

Treatment of compounds of general Formula XVI (Scheme 6) with ammonia gives compounds of general Formula XXI. In some cases, the addition of and oxidizing reagent such as tert-butyl hydroperoxide might be necessary to facilitate the reaction. Compounds of general Formula I may be prepared by reacting compounds of general Formula XXI with a carboxylic acid chloride of Formula XXII or by reaction with a carboxylic acid of Formula XXIII using procedures known to chemists skilled in the art.

### PREPARATION OF INTERMEDIATES

### Intermediate: (R)-N-(1-(2-Fluorophenyl)ethylidene)-2-methylpropane-2-sulfinamide

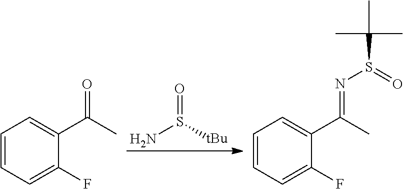

To a solution of 1-(2-fluorophenyl)ethanone (25.0 g, 181 mmol) in THF (500 mL) was added Ti(OEt)₄ (82.6 g, 362.0 mmol) and (R)-2-methylpropane-2-sulfinamide (26.3 g, 217 mmol). The mixture was stirred at 85° C. for 14 hours. The reaction mixture was quenched with water (200 mL) and then filtered and the filtrate was extracted with ethyl acetate (3×200 mL). The combined organic layers were washed with brine (100 mL), dried over Na₂SO₄, filtered and concentrated under reduced pressure. The residue was combined with four similar batches (25 g reactant 1-(2-fluorophenyl)ethanone for each batch), the mixture was purified by column chromatography (silica gel, petroleum ether/ethyl acetate=10:1 to 5:1). (R)-N-(1-(2-fluorophenyl)ethylidene)-2-methylpropane-2-sulfinamide (135.0 g, 77.3% yield) was obtained.

(R)-N-(1-(2,3-Difluorophenyl)ethylidene)-2-methylpropane-2-sulfinamide was prepared in a similar way from 1-(2,3-difluorophenyl)ethan-1-one.

(R)-N-(2-Fluoro-1-(2-fluorophenyl)ethylidene)-2-methylpropane-2-sulfinamide was prepared in a similar way from 2-fluoro-1-(2-fluorophenyl)ethan-1-one.

### Intermediate: 2-(2-Fluorophenyl)-2,2-dimethoxyethan-1-ol

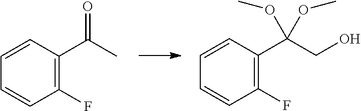

To a mixture of potassium hydroxide (91.4 g, 1.63 mol) in methanol (1 L) was added a solution of 1-(2-fluorophenyl)ethan-1-one (50 g, 360 mmol) in methanol (300 mL) in a dropwise manner at 0° C. Then bis(acetoxy)iodobenzene (175 g, 543 mmol) was added in portions. After stirring at 0° C. for 4 hours, the reaction was quenched with the addition of water (500 mL). The mixture was concentrated to remove methanol and the aqueous phase was extracted with ethyl acetate (700 mL, three times), the combined organic phases were washed with brine (300 mL), dried over Na₂SO₄ and concentrated. The crude product was used in the next step directly without further purification.

### Intermediate: 1-(2-Fluorophenyl)-2-hydroxyethan-1-one

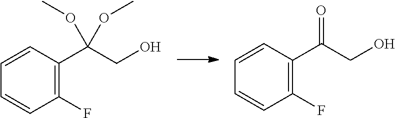

2-(2-Fluorophenyl)-2,2-dimethoxyethan-1-ol (360 mmol) was dissolved in THF (450 mL) and water (150 mL). Then p-toluene sulfonic acid (125 g, 726 mmol) was added portionwise at room temperature. After the addition, the mixture was stirred under reflux for 5 hours. Water (150 mL) and sat. NaHCO₃ was added to quench the reaction, the mixture was extracted with ethyl acetate (500 mL, three times). The combined organic layers were washed with brine and dried over Na₂SO₄. After removal of the solvents under reduced pressure, the residue was purified by column chromatography with petroleum ether: ethyl acetate=20:1 to give 1-(2-fluorophenyl)-2-hydroxyethan-1-one (42 g, 76% yield, two steps).

### Intermediate: 2-Fluoro-1-(2-fluorophenyl)ethan-1-one

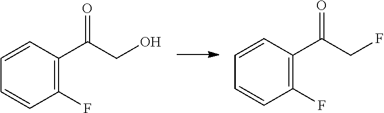 [SMILES: O=C(CO)c1ccccc1F ; O=C(CF)c1ccccc1F]

To a solution of 1-(2-fluorophenyl)-2-hydroxyethan-1-one (10 g, 64.9 mmol) in dichloromethane (200 mL) was added Et₃N(HF)₃ (10.46 g, 227.8 mmol) and CF₃(CF₂)₃SO₂F (29.4 g, 97.32 mmol) dropwise at 0° C., the solution was stirred at room temperature for 12 hours. TLC (petroleum ether: ethyl acetate=10:1) showed no starting material. The mixture was poured into a saturated solution of NaHCO₃ and ice, extracted with dichloromethane (200 mL three times), the combined organic layers were washed with brine, then dried over Na₂SO₄ and concentrated under reduced pressure. The mixture was purified by column chromatography on silica gel (eluted with petroleum ether: ethyl acetate=1:0-10:1) to afford 2-fluoro-1-(2-fluorophenyl)ethan-1-one (6 g, yield: 59%).

### Intermediate: Ethyl (S)-3-(((R)-tert-butylsulfinyl)amino)-3-(2-fluorophenyl)butanoate

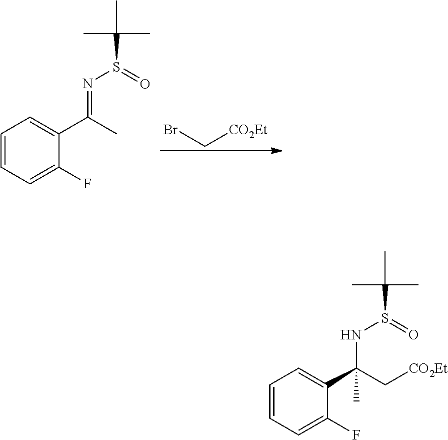 [SMILES: C/C(=N\[S@](=O)C(C)(C)C)c1ccccc1F ; CCOC(=O)CBr ; CCOC(=O)C[C@](C)(N[S@](=O)C(C)(C)C)c1ccccc1F]

To a solution of (R)-N-(1-(2-fluorophenyl)ethylidene)-2-methylpropane-2-sulfinamide (20.0 g, 82.9 mmol) and Rh(PPh₃)₃Cl (2.3 g, 2.5 mmol) in dry THF (300 mL) was added dropwise Et₂Zn (1 M, 162 mL) at −78° C. The mixture was stirred at −78° C.-0° C. for 2 hours. The reaction mixture was quenched by NH₄Cl (sat. aq. 100 mL) at 0° C., and then diluted with water (200 mL) and extracted with ethyl acetate (2×200 mL). The combined organic layers were washed with brine (50 mL) dried over Na₂SO₄, filtered and concentrated under reduced pressure. The crude product was purified by flash chromatography on silica gel with petroleum ether/ethyl acetate=4:1. Ethyl (S)-3-(((R)-tert-butylsulfinyl)amino)-3-(2-fluorophenyl)butanoate (23.3 g, 85.3% yield) was obtained as a yellow oil. ¹H NMR (CDCl₃ 400 MHz): δ 7.53-7.49 (m, 1H), 7.27-7.25 (m, 1H), 7.14-7.12 (m, 1H), 7.05-6.99 (m, 1H), 5.15 (s, 1H), 4.02 (q, J=7.2 Hz, 2H), 3.38 (d, J=16.0 Hz, 1H), 3.10 (d, J=17.6 Hz, 1H), 1.85 (s, 3H), 1.31 (s, 9H), 1.13 (t, J=7.2 Hz, 3H).

Ethyl (S)-3-(((R)-tert-butylsulfinyl)amino)-3-(2,3-difluorophenyl)butanoate was prepared in a similar way from (R)-N-(1-(2,3-difluorophenyl)ethylidene)-2-methylpropane-2-sulfinamide.

Ethyl (S)-3-(((R)-tert-butylsulfinyl)amino)-4-fluoro-3-(2-fluorophenyl)butanoate was prepared in a similar way from ((R)-N-(2-fluoro-1-(2-fluorophenyl)ethylidene)-2-methylpropane-2-sulfinamide.

### Intermediate: (S)-3-(((R)-tert-Butylsulfinyl)amino)-3-(2-fluorophenyl)butanoic acid

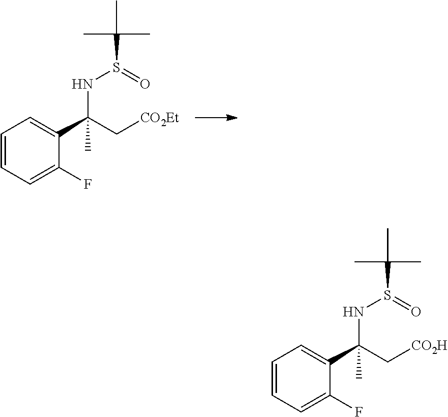 [SMILES: CCOC(=O)C[C@](C)(N[S@](=O)C(C)(C)C)c1ccccc1F ; CC(C)(C)[S@@](=O)N[C@@](C)(CC(=O)O)c1ccccc1F]

To a solution of ethyl (S)-3-(((R)-tert-butylsulfinyl)amino)-3-(2-fluorophenyl)butanoate (18.3 g, 55.6 mmol) in THF (180 mL) and H₂O (60 mL) was added LiOH.H₂O (2.8 g, 66.7 mmol). The mixture was stirred at room temperature for 18 hours. The reaction mixture was adjusted pH to 3-4 by adding KHSO₄ (sat. aq.). The resulting mixture was extracted with ethyl acetate (3×200 mL). The combined organic layers were washed with brine (50 mL), dried over Na₂SO₄ and concentrated. (S)-3-(((R)-tert-butylsulfinyl)amino)-3-(2-fluorophenyl)butanoic acid (16.7 g, crude) was obtained and was used into the next step without further purification.

(S)-3-(((R)-tert-Butylsulfinyl)amino)-3-(2,3-difluorophenyl)butanoic acid was prepared in a similar way from ethyl (S)-3-(((R)-tert-butylsulfinyl)amino)-3-(2,3-difluorophenyl)butanoate.

(S)-3-(((R)-tert-butylsulfinyl)amino)-4-fluoro-3-(2-fluorophenyl)butanoic acid was prepared in a similar way from ethyl (S)-3-(((R)-tert-butylsulfinyl)amino)-4-fluoro-3-(2-fluorophenyl)butanoate.

### Intermediate: (R)-N-((S)-4-(2,2-Dimethyl-4,6-dioxo-1,3-dioxan-5-yl)-2-(2-fluorophenyl)-4-oxobutan-2-yl)-2-methylpropane-2-sulfinamide

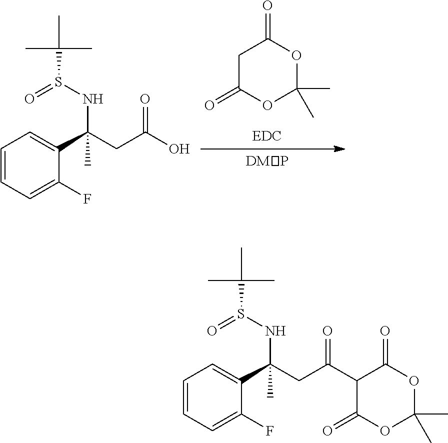

To a solution of (S)-3-(((R)-tert-butylsulfinyl)amino)-3-(2-fluorophenyl)butanoic acid (10.0 g, 33.2 mmol) in dry THF (100 mL) was added 1-ethyl-3-(3-dimethylaminopropyl)carbodiimide (EDC) (7.6 g, 39.8 mmol) and 4-dimethylaminopyridine (DMAP) (4.9 g, 39.8 mmol). The mixture was stirred at room temperature for 5 min, then Meldrum's acid (2,2-dimethyl-1,3-dioxane-4,6-dione) (4.8 g, 33.2 mmol) was added. The mixture was stirred at room temperature for 18 hours. Water (150 mL) was added, the resulting mixture was extracted with ethyl acetate (3×150 mL). The combined organic layers were washed with brine (50 mL), dried over Na₂SO₄ and concentrated. (R)-N-((S)-4-(2,2-dimethyl-4,6-dioxo-1,3-dioxan-5-yl)-2-(2-fluorophenyl)-4-oxobutan-2-yl)-2-methylpropane-2-sulfinamide (14.2 g, crude) was obtained and was used into the next step without further purification.

(R)-N-((S)-2-(2,3-Difluorophenyl)-4-(2,2-dimethyl-4,6-dioxo-1,3-dioxan-5-yl)-4-oxobutan-2-yl)-2-methylpropane-2-sulfinamide was prepared in a similar way from (S)-3-(((R)-tert-butylsulfinyl)amino)-3-(2,3-difluorophenyl)butanoic acid.

(R)-N-((S)-4-(2,2-dimethyl-4,6-dioxo-1,3-dioxan-5-yl)-1-fluoro-2-(2-fluorophenyl)-4-oxobutan-2-yl)-2-methylpropane-2-sulfinamide was prepared in a similar way from (S)-3-(((R)-tert-butylsulfinyl)amino)-4-fluoro-3-(2-fluorophenyl)butanoic acid.

### Intermediate: (R)-N-((S)-4-(2,2-Dimethyl-4,6-dioxo-1,3-dioxan-5-yl)-2-(2-fluorophenyl)butan-2-yl)-2-methylpropane-2-sulfinamide

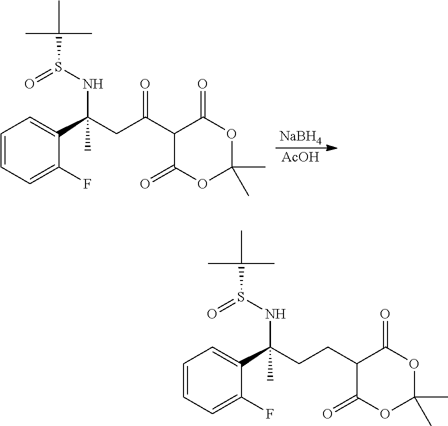

To a solution of (R)-N-((S)-4-(2,2-dimethyl-4,6-dioxo-1,3-dioxan-5-yl)-2-(2-fluorophenyl)-4-oxobutan-2-yl)-2-methylpropane-2-sulfinamide (14.2 g, 33.2 mmol) in acetic acid (150 mL) was added NaBH₄ (6.3 g, 165.9 mmol) in 5 portions with 15 minute intervals. Then the mixture was stirred at room temperature for 18 hours. NH₄Cl (sat. aq. 100 mL) was added to quench the reaction, then the mixture was concentrated. Water (150 mL) was added and the mixture was extracted with ethyl acetate (3×170 mL), the combined organic layers were washed with brine (100 mL), dried over Na₂SO₄ and concentrated. (R)-N-((S)-4-(2,2-dimethyl-4,6-dioxo-1,3-dioxan-5-yl)-2-(2-fluorophenyl)butan-2-yl)-2-methylpropane-2-sulfinamide (13.7 g, crude) was obtained and was used into the next step without further purification.

(R)-N-((S)-2-(2,3-Difluorophenyl)-4-(2,2-dimethyl-4,6-dioxo-1,3-dioxan-5-yl)butan-2-yl)-2-methylpropane-2-sulfinamide was prepared in a similar way from (R)-N-((S)-2-(2,3-difluorophenyl)-4-(2,2-dimethyl-4,6-dioxo-1,3-dioxan-5-yl)-4-oxobutan-2-yl)-2-methylpropane-2-sulfinamide.

(R)-N-((S)-4-(2,2-dimethyl-4,6-dioxo-1,3-dioxan-5-yl)-1-fluoro-2-(2-fluorophenyl)butan-2-yl)-2-methylpropane-2-sulfinamide was prepared in a similar way from (R)-N-((S)-4-(2,2-dimethyl-4,6-dioxo-1,3-dioxan-5-yl)-1-fluoro-2-(2-fluorophenyl)-4-oxobutan-2-yl)-2-methylpropane-2-sulfinamide.

### Intermediate: (R)-N-((S)-4-(5-Fluoro-2,2-dimethyl-4,6-dioxo-1,3-dioxan-5-yl)-2-(2-fluorophenyl)butan-2-yl)-2-methylpropane-2-sulfinamide

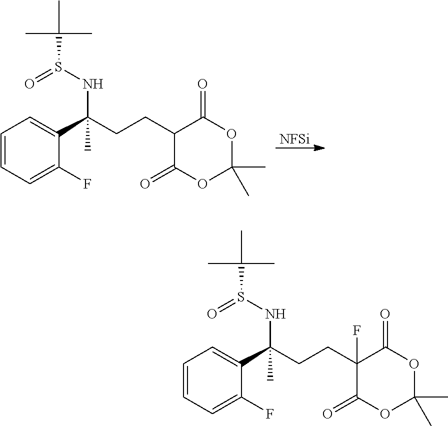

To a solution of (R)-N-((S)-4-(2,2-dimethyl-4,6-dioxo-1,3-dioxan-5-yl)-2-(2-fluorophenyl)butan-2-yl)-2-methylpropane-2-sulfinamide (5.0 g, 12.1 mmol) in anhydrous THF (75 mL) was added N-fluorodibenzenesulfonamide (NFSi) (4.6 g, 14.5 mmol) and 1,8-diazabicyclo[5.4.0]undec-7-ene (DBU) (1.8 g, 12.1 mmol) at 0° C. The mixture was stirred at 0° C.—room temperature for 1 hour. The mixture was filtered and the filtrate was concentrated. (R)-N-((S)-4-(5-fluoro-2,2-dimethyl-4,6-dioxo-1,3-dioxan-5-yl)-2-(2-fluorophenyl)butan-2-yl)-2-methylpropane-2-sulfinamide (5.2 g, crude) was obtained and was used into the next step without further purification.

(R)-N-((S)-2-(2,3-Difluorophenyl)-4-(5-fluoro-2,2-dimethyl-4,6-dioxo-1,3-dioxan-5-yl)butan-2-yl)-2-methylpropane-2-sulfinamide was prepared in a similar way from (R)-N-((S)-2-(2,3-difluorophenyl)-4-(2,2-dimethyl-4,6-dioxo-1,3-dioxan-5-yl)butan-2-yl)-2-methylpropane-2-sulfinamide.

(R)-N-((S)-1-fluoro-4-(5-fluoro-2,2-dimethyl-4,6-dioxo-1,3-dioxan-5-yl)-2-(2-fluorophenyl)butan-2-yl)-2-methylpropane-2-sulfinamide was prepared in a similar way from (R)-N-((S)-4-(2,2-dimethyl-4,6-dioxo-1,3-dioxan-5-yl)-1-fluoro-2-(2-fluorophenyl)butan-2-yl)-2-methylpropane-2-sulfinamide.

### Intermediate: Methyl (6S)-3-fluoro-6-(2-fluorophenyl)-6-methyl-2-oxopiperidine-3-carboxylate

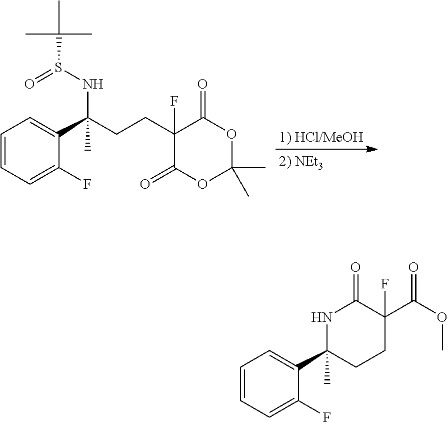

To a solution of (R)-N-((S)-4-(5-fluoro-2,2-dimethyl-4,6-dioxo-1,3-dioxan-5-yl)-2-(2-fluorophenyl)butan-2-yl)-2-methylpropane-2-sulfinamide (5.2 g, 12.1 mmol) in MeOH (50 mL) was added HCl/MeOH (4 M, 30.3 mL). The mixture was stirred at room temperature for 18 hours. The mixture was concentrated in vacuo and redissolved in MeOH (60 mL). Triethylamine was added (2.9 g, 29.1 mmol). The mixture was stirred at 80° C. for 18 hours. The mixture was concentrated. Then anhydrous THF (100 mL) was added and the mixture was stirred at room temperature for 5 minutes. The mixture was filtered and the residue was washed with anhydrous THF (2×30 mL) and the combined filtrates were concentrated. Methyl (6S)-3-fluoro-6-(2-fluorophenyl)-6-methyl-2-oxopiperidine-3-carboxylate (3.4 g) was obtained and was used into the next step without further purification.

Methyl (6S)-6-(2,3-difluorophenyl)-3-fluoro-6-methyl-2-oxopiperidine-3-carboxylate was prepared in a similar way from (R)-N-((S)-2-(2,3-difluorophenyl)-4-(5-fluoro-2,2-dimethyl-4,6-dioxo-1,3-dioxan-5-yl)butan-2-yl)-2-methylpropane-2-sulfinamide.

Methyl (6S)-3-fluoro-6-(fluoromethyl)-6-(2-fluorophenyl)-2-oxopiperidine-3-carboxylate was prepared in a similar way from (R)-N-((S)-1-fluoro-4-(5-fluoro-2,2-dimethyl-4,6-dioxo-1,3-dioxan-5-yl)-2-(2-fluorophenyl)butan-2-yl)-2-methylpropane-2-sulfinamide.

### Intermediate: (6S)-3-Fluoro-6-(2-fluorophenyl)-3-(hydroxymethyl)-6-methylpiperidin-2-one

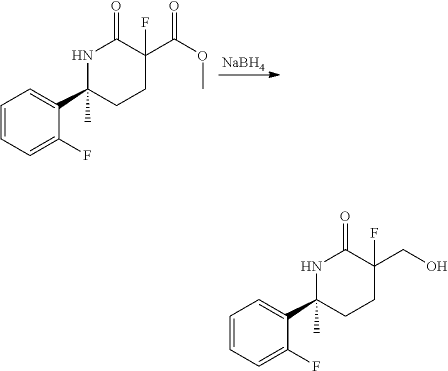

To a solution of methyl (6S)-3-fluoro-6-(2-fluorophenyl)-6-methyl-2-oxopiperidine-3-carboxylate (3.4 g, 12.1 mmol) in MeOH (50 mL) was added NaBH₄ (3.7 g, 96.9 mmol) in 5 portions with 15 minute intervals. The mixture was stirred at room temperature for 17 hours. The mixture was concentrated, then water (200 mL) was added and the mixture was extracted with ethyl acetate (3×150 mL). The combined organic layers were washed with brine (50 mL), dried over Na₂SO₄ and concentrated. The crude product was purified by flash chromatography on silica gel with petroleum ether:ethyl acetate=1:1-1:2 to give (6S)-3-fluoro-6-(2-fluorophenyl)-3-(hydroxymethyl)-6-methylpiperidin-2-one (2.5 g, 81% yield).

(6S)-6-(2,3-Difluorophenyl)-3-fluoro-3-(hydroxymethyl)-6-methylpiperidin-2-one was prepared in a similar way from methyl (6S)-6-(2,3-difluorophenyl)-3-fluoro-6-methyl-2-oxopiperidine-3-carboxylate.

(6S)-3-Fluoro-6-(fluoromethyl)-6-(2-fluorophenyl)-3-(hydroxymethyl)piperidin-2-one was prepared in a similar way from methyl (6S)-3-fluoro-6-(fluoromethyl)-6-(2-fluorophenyl)-2-oxopiperidine-3-carboxylate.

### Intermediate: (6S)-3-Fluoro-3-(fluoromethyl)-6-(2-fluorophenyl)-6-methylpiperidin-2-one

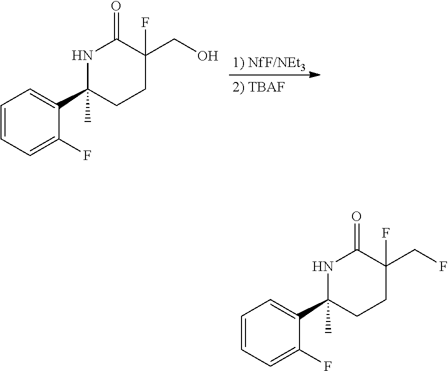

To a solution of (6S)-3-fluoro-6-(2-fluorophenyl)-3-(hydroxymethyl)-6-methylpiperidin-2-one (2.8 g, 11.0 mmol) in anhydrous THF (60 mL) was added nonafluorobutanesulfonyl fluoride (NfF) (9.9 g, 32.9 mmol) and triethylamine (4.4 g, 43.9 mmol). The mixture was stirred at room temperature for 2.5 hours. A solution of tetra-N-butylammonium fluoride (TBAF) (1 M, 13.2 mL) in THF was added. The mixture was stirred at 50° C. for 16 hours. Water (150 mL) was added. The mixture was extracted with ethyl acetate (3×100 mL). The combined organic phase was washed with brine (30 mL), dried over MgSO₄ and concentrated in vacuo. The crude material was purified via flash chromatography on silica gel with petroleum ether:ethyl acetate=2:1 to give (6S)-3-fluoro-3-(fluoromethyl)-6-(2-fluorophenyl)-6-methylpiperidin-2-one (2.4 g, 85% yield).

(6S)-6-(2,3-Difluorophenyl)-3-fluoro-3-(fluoromethyl)-6-methylpiperidin-2-one was prepared in a similar way from (6S)-6-(2,3-difluorophenyl)-3-fluoro-3-(hydroxymethyl)-6-methylpiperidin-2-one.

(6S)-3-Fluoro-3,6-bis(fluoromethyl)-6-(2-fluorophenyl)piperidin-2-one was prepared in a similar way from (6S)-3-fluoro-6-(fluoromethyl)-6-(2-fluorophenyl)-3-(hydroxymethyl)piperidin-2-one.

### Intermediates: (3S,6S)-3-Fluoro-6-(2-fluoro-5-nitrophenyl)-3-(fluoromethyl)-6-methylpiperidin-2-one and (3R,6S)-3-fluoro-6-(2-fluoro-5-nitrophenyl)-3-(fluoromethyl)-6-methylpiperidin-2-one

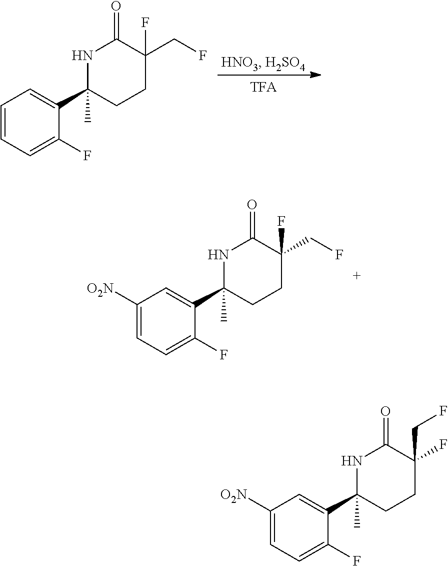

(6S)-3-Fluoro-3-(fluoromethyl)-6-(2-fluorophenyl)-6-methylpiperidin-2-one (1.4 g, 5.4 mmol) was suspended in TFA (9 mL). The mixture was cooled to 0° C. and concentrated H₂SO₄ (4.2 g, 41.9 mmol) was added. Finally, HNO₃ (1.7 g, 16.3 mmol) (60%) was added dropwise. After addition, the black brown mixture was stirring at 0° C. for 2 hours. The mixture was poured onto 100 g ice and basified to pH>11 using 5 M NaOH (aq.). The suspension was extracted with ethyl acetate (150 mL). The phases were separated and the aqueous layer was extracted with ethyl acetate (2×100 mL). The combined organics were washed with a solution of saturated aqueous NH₄Cl (50 mL) and water (50 mL), dried over MgSO₄, filtered, and concentrated under reduced pressure. The residue was purified by column chromatography (silica gel, petroleum ether/ethyl acetate=2/1) to give (3S,6S)-3-fluoro-6-(2-fluoro-5-nitrophenyl)-3-(fluoromethyl)-6-methylpiperidin-2-one and (3R,6S)-3-fluoro-6-(2-fluoro-5-nitrophenyl)-3-(fluoromethyl)-6-methylpiperidin-2-one.

(3S,6S)-3-Fluoro-6-(2-fluoro-5-nitrophenyl)-3-(fluoromethyl)-6-methylpiperidin-2-one (900 mg)¹H NMR (CDCl₃ 400 MHz): δ 8.42-8.40 (m, 1H), 8.26-8.22 (m, 1H), 7.56 (brs, 1H), 7.31-7.26 (m, 1H), 4.78 (d, J=19.6 Hz, 1H), 4.67 (d, J=19.6 Hz, 1H), 2.61-2.57 (m, 1H), 2.38-2.30 (m, 1H), 2.20-2.19 (m, 1H), 2.19-2.16 (m, 1H), 1.78 (s, 3H).

(3R,6S)-3-Fluoro-6-(2-fluoro-5-nitrophenyl)-3-(fluoromethyl)-6-methylpiperidin-2-one (600 mg)¹H NMR (CDCl₃ 400 MHz): δ 8.29-8.22 (m, 2H), 7.72 (brs, 1H), 7.31-7.26 (m, 1H), 4.72-4.47 (m, 2H), 2.62-2.58 (m, 1H), 2.30-2.25 (m, 2H), 2.19-1.80 (s, 3H), 1.80-1.66 (m, 1H).

(3S,6S)-6-(2,3-Difluoro-5-nitrophenyl)-3-fluoro-3-(fluoromethyl)-6-methylpiperidin-2-one and (3R,6S)-6-(2,3-difluoro-5-nitrophenyl)-3-fluoro-3-(fluoromethyl)-6-methylpiperidin-2-one were prepared in a similar way from (6S)-6-(2,3-difluorophenyl)-3-fluoro-3-(fluoromethyl)-6-methylpiperidin-2-one.

(6S)-3-Fluoro-6-(2-fluoro-5-nitrophenyl)-3,6-bis(fluoromethyl)piperidin-2-one was prepared in a similar way from (6S)-3-fluoro-3,6-bis(fluoromethyl)-6-(2-fluorophenyl)piperidin-2-one.

### Intermediate: (3R,6S)-3-Fluoro-6-(2-fluoro-5-nitrophenyl)-3-(fluoromethyl)-6-methylpiperidine-2-thione

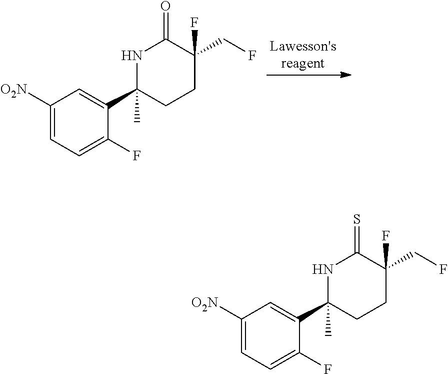 [SMILES: C[C@@]1(c2cc([N+](=O)[O-])ccc2F)CC[C@@](F)(CF)C(=O)N1 ; "Lawesson'sreagent" ; C[C@@]1(c2cc([N+](=O)[O-])ccc2F)CC[C@@](F)(CF)C(=S)N1]

To a solution of (3S,6S)-3-fluoro-6-(2-fluoro-5-nitrophenyl)-3-(fluoromethyl)-6-methylpiperidin-2-one (900 mg, 3.0 mmol) in anhydrous toluene (18 mL) was added 2,4-bis(4-methoxyphenyl)-1,3,2,4-dithiadiphosphetane-2,4-dithione (Lawesson's reagent) (723 mg, 1.8 mmol). The mixture was stirred at 80° C. for 2 hours. The mixture was concentrated. The crude product was purified by flash chromatography with petroleum ether:ethyl acetate=3:1 to give (3R,6S)-3-fluoro-6-(2-fluoro-5-nitrophenyl)-3-(fluoromethyl)-6-methylpiperidine-2-thione (900 mg, 95% yield).

(3S,6S)-3-fluoro-6-(2-fluoro-5-nitrophenyl)-3-(fluoromethyl)-6-methylpiperidine-2-thione was prepared in a similar way from (3R,6S)-3-fluoro-6-(2-fluoro-5-nitrophenyl)-3-(fluoromethyl)-6-methylpiperidin-2-one.

(3R,6S)-6-(2,3-Difluoro-5-nitrophenyl)-3-fluoro-3-(fluoromethyl)-6-methylpiperidine-2-thione was prepared in a similar way from (3S,6S)-6-(2,3-difluoro-5-nitrophenyl)-3-fluoro-3-(fluoromethyl)-6-methylpiperidin-2-one.

(3S,6S)-6-(2,3-Difluoro-5-nitrophenyl)-3-fluoro-3-(fluoromethyl)-6-methylpiperidine-2-thione was prepared in a similar way from (3R,6S)-6-(2,3-difluoro-5-nitrophenyl)-3-fluoro-3-(fluoromethyl)-6-methylpiperidin-2-one.

(6S)-3-Fluoro-6-(2-fluoro-5-nitrophenyl)-3,6-bis(fluoromethyl)piperidine-2-thione was prepared in a similar way from (6S)-3-fluoro-6-(2-fluoro-5-nitrophenyl)-3,6-bis(fluoromethyl)piperidin-2-one.

### Intermediate: (3R,6S)-6-(5-Amino-2-fluorophenyl)-3-fluoro-3-(fluoromethyl)-6-methylpiperidine-2-thione

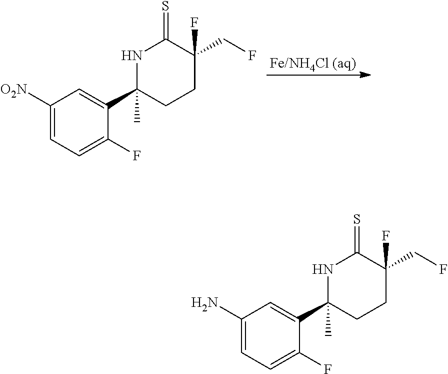

To a solution of (3R,6S)-3-fluoro-6-(2-fluoro-5-nitrophenyl)-3-(fluoromethyl)-6-methylpiperidine-2-thione (900 mg, 2.8 mmol) in EtOH (18.0 mL) and H₂O (4.5 mL) was added Fe (790 mg, 14.15 mmol) and NH₄Cl (756 mg, 14.2 mmol). The mixture was stirred at room temperature for 5 hours. The mixture was filtered and the residue was washed with EtOH (30 mL). The combined filtrates were concentrated. The residue was dispersed in ethyl acetate (30 mL) and then filtered. The filter cake was washed with ethyl acetate (2×15 mL). The combined organic layers were concentrated. The crude product was purified by flash chromatography with petroleum ether:ethyl acetate=3:1 to give (3R,6S)-6-(5-amino-2-fluorophenyl)-3-fluoro-3-(fluoromethyl)-6-methylpiperidine-2-thione (712 mg, 87% yield). ¹H NMR (600 MHz, CDCl₃) δ 8.42 (s, 1H), 6.88 (dd, J=11.8, 8.6 Hz, 1H), 6.58 (dt, J=7.0, 3.2 Hz, 1H), 6.50 (dd, J=6.8, 2.8 Hz, 1H), 4.95 (ddd, J=49.0, 12.6, 10.9 Hz, 1H), 4.61 (ddd, J=46.7, 28.4, 10.7 Hz, 1H), 3.70 (s, 2H), 2.62-2.54 (m, 1H), 2.39-2.30 (m, 1H), 2.10-2.02 (m, 1H), 1.91 (q, J=14.0 Hz, 1H), 1.70 (s, 3H). [α]_{D}²⁰=−236° (c=0.10, EtOH).

(3S,6S)-6-(5-Amino-2-fluorophenyl)-3-fluoro-3-(fluoromethyl)-6-methylpiperidine-2-thione was prepared in a similar way from (3S,6S)-3-fluoro-6-(2-fluoro-5-nitrophenyl)-3-(fluoromethyl)-6-methylpiperidine-2-thione. ¹H NMR (600 MHz, CDCl₃) δ 8.58 (s, 1H), 6.88 (dd, J=11.7, 8.6 Hz, 1H), 6.58 (dt, J=8.4, 3.2 Hz, 1H), 6.38 (dd, J=6.7, 2.7 Hz, 1H), 4.97 (dt, J=48.8, 10.6 Hz, 1H), 4.60 (ddd, J=46.0, 24.6, 10.3 Hz, 1H), 3.68 (s, 2H), 2.53 (d, J=14.1 Hz, 1H), 2.26-2.17 (m, 1H), 2.13 (td, J=13.9, 2.6 Hz, 1H), 1.87-1.74 (m, 1H), 1.73 (s, 3H). [α]_{D}²⁰=−154° (c=0.10, EtOH).

(3R,6S)-6-(5-Amino-2,3-difluorophenyl)-3-fluoro-3-(fluoromethyl)-6-methylpiperidine-2-thione was prepared in a similar way from (3R,6S)-6-(2,3-difluoro-5-nitrophenyl)-3-fluoro-3-(fluoromethyl)-6-methylpiperidine-2-thione.

(3S,6S)-6-(5-Amino-2,3-difluorophenyl)-3-fluoro-3-(fluoromethyl)-6-methylpiperidine-2-thione was prepared in a similar way from (3S,6S)-6-(2,3-difluoro-5-nitrophenyl)-3-fluoro-3-(fluoromethyl)-6-methylpiperidine-2-thione.

(3R,6S)-6-(5-Amino-2-fluorophenyl)-3-fluoro-3,6-bis(fluoromethyl)piperidine-2-thione and (3S,6S)-6-(5-amino-2-fluorophenyl)-3-fluoro-3,6-bis(fluoromethyl)piperidine-2-thione were prepared in a similar way starting from (6S)-3-fluoro-6-(2-fluoro-5-nitrophenyl)-3,6-bis(fluoromethyl)piperidine-2-thione followed by chromatographic separation of the two diastereomers.

(3R,6S)-6-(5-Amino-2-fluorophenyl)-3-fluoro-3,6-bis(fluoromethyl)piperidine-2-thione: ¹H NMR (CDCl₃ 400 MHz) δ 8.42 (s, 1H), 6.95-6.89 (m, 1H), 6.65-6.62 (m, 1H), 6.54 (dd, J=6.8, 3.2 Hz, 1H), 5.03-4.51 (m, 4H), 3.73 (s, 2H), 2.48-2.39 (m, 2H), 2.22 (t, J=14.0 Hz, 1H), 1.96 (q, J=12.8 Hz, 1H). [α]_{D}²⁰=−204° (c=0.10, EtOH).

(3S,6S)-6-(5-Amino-2-fluorophenyl)-3-fluoro-3,6-bis(fluoromethyl)piperidine-2-thione: ¹H NMR (CDCl₃ 400 MHz) δ 8.65 (s, 1H), 6.95-6.90 (m, 1H), 6.64-6.62 (m, 1H), 6.41 (dd, J=6.8, 2.8 Hz, 1H), 5.09-4.95 (m, 2H), 4.89-4.52 (m, 2H), 2.41-2.38 (m, 1H), 2.33-2.24 (m, 1H), 2.21-2.14 (m, 1H), 1.92-1.75 (m, 1H). [α]_{D}²⁰=−130° (c=0.10, EtOH).

### Preparation of N-[3-[(2S,5R)-6-amino-5-fluoro-5-(fluoromethyl)-2-methyl-2,3,4,5-tetrahydropyridin-2-yl]-4,5-difluoro-phenyl]-5-methoxy-pyrazine-2-carboxamide

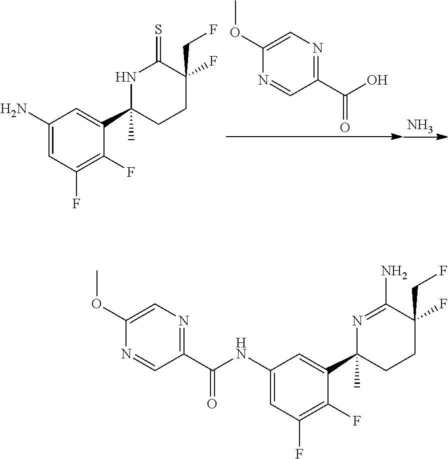

A solution of (3S,6S)-6-(5-amino-2,3-difluorophenyl)-3-fluoro-3-(fluoromethyl)-6-methylpiperidine-2-thione (200 mg, 0.60 mmol), 5-methoxypyrazine-2-carboxylic acid (139 mg, 901 micromole), HATU (1-[bis(dimethylamino)methylene]-1H-1,2,3-triazolo[4,5-b]pyridinium 3-oxide hexafluorophosphate) (297 mg, 781 micromole), DIPEA (N,N-diisopropylethylamine) (388 mg, 3.0 mmol, 0.53 mL) in DMF (8.0 mL) was stirred at 20° C. for 3.0 hours under an atmosphere of nitrogen. The reaction mixture was quenched with saturated aqueous NH₄Cl (10 mL), and then diluted with ethyl acetate (20 mL) and extracted with ethyl acetate (15 mL×3). The combined organic layers were washed with brine (15 mL), dried over Na₂SO₄, filtered and concentrated under reduced pressure to give crude N-(3,4-difluoro-5-((2S,5S)-5-fluoro-5-(fluoromethyl)-2-methyl-6-thioxopiperidin-2-yl)phenyl)-5-methoxypyrazine-2-carboxamide (266 mg) which was used into the next step without further purification. A mixture of crude N-(3,4-difluoro-5-((2S,5S)-5-fluoro-5-(fluoromethyl)-2-methyl-6-thioxopiperidin-2-yl)phenyl)-5-methoxypyrazine-2-carboxamide (266 mg) from the previous step, 2-hydroperoxy-2-methyl-propane (5.5 M in decane, 0.20 mL) in NH₃ (7.0 M in methanol, 20 mL) was stirred at 50° C. for 15 hours under an atmosphere of nitrogen. The mixture was concentrated and purified by preparative HPLC to give N-[3-[(2S,5R)-6-amino-5-fluoro-5-(fluoromethyl)-2-methyl-2,3,4,5-tetrahydropyridin-2-yl]-4,5-difluoro-phenyl]-5-methoxy-pyrazine-2-carboxamide (40 mg).

¹H NMR (400 MHz, CDCl₃): δ 9.51 (s, 1H), 9.00 (d, J=1.2 Hz, 1H), 8.16 (d, J=1.2 Hz, 1H), 7.98-7.93 (m, 1H), 7.16 (dt, J=5.6, 2.4 Hz, 1H), 4.70-4.43 (m, 2H), 4.08 (s, 3H), 2.80 (brs, 2H), 2.20 (dd, J=8.4, 4.0 Hz, 2H), 2.15-2.09 (m, 1H), 1.94-1.82 (m, 1H), 1.68 (s, 3H). [α]_{D}²⁰=−9.00 (589 nm, c=0.10, MeOH).

The following intermediates were prepared in a similar manner:
N-(3-((2S,5S)-6-Amino-5-fluoro-5-(fluoromethyl)-2-methyl-2,3,4,5-tetrahydropyridin-2-yl)-4-fluorophenyl)-5-methoxypyrazine-2-carboxamidepreparation

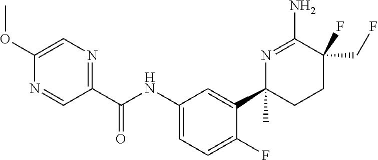 [SMILES: COc1cnc(C(=O)Nc2ccc(F)c([C@]3(C)CC[C@@](F)(CF)C(N)=N3)c2)cn1]

Prepared from (3R,6S)-6-(5-amino-2-fluorophenyl)-3-fluoro-3-(fluoromethyl)-6-methylpiperidine-2-thione and 5-methoxypyrazine-2-carboxylic acid ¹H NMR (600 MHz, DMSO) δ 10.45 (s, 1H), 8.88 (d, J=1.0 Hz, 1H), 8.41 (d, J=1.0 Hz, 1H), 7.83 (dd, J=7.3, 2.4 Hz, 1H), 7.78-7.74 (m, 1H), 7.13 (dd, J=11.8, 8.8 Hz, 1H), 6.12 (s, 2H), 4.96-4.68 (m, 2H), 4.02 (s, 3H), 2.26-2.04 (m, 2H), 1.96-1.89 (m, 1H), 1.64-1.53 (m, 1H), 1.46 (s, 3H).

### N-[3-[(2S,5S)-6-Amino-5-fluoro-5-(fluoromethyl)-2-methyl-2,3,4,5-tetrahydropyridin-2-yl]-4,5-difluoro-phenyl]-5-methoxy-pyrazine-2-carboxamide

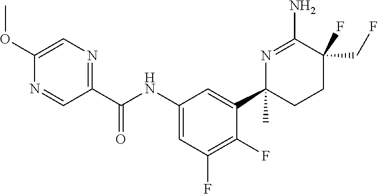 [SMILES: COc1cnc(C(=O)Nc2cc(F)c(F)c([C@]3(C)CC[C@@](F)(CF)C(N)=N3)c2)cn1]

Prepared from (3R,6S)-6-(5-amino-2,3-difluorophenyl)-3-fluoro-3-(fluoromethyl)-6-methylpiperidine-2-thione and 5-methoxypyrazine-2-carboxylic acid ¹H NMR (400 MHz, CDCl₃): δ 9.54 (s, 1H), 9.00 (d, J=1.6 Hz, 1H), 9.15 (d, J=1.6 Hz, 1H), 8.13-8.07 (m, 1H), 7.14 (dt, J=6.0, 2.0 Hz, 1H), 4.81-4.56 (m, 2H), 4.08 (s, 3H), 3.05 (brs, 2H), 2.53-2.46 (m, 1H), 2.20-2.13 (m, 1H), 1.94 (td, J=13.6, 2.4 Hz, 1H), 1.82-1.70 (m, 1H), 1.64 (d, J=1.2 Hz, 3H). [α]_{D}²⁰=+12.0 (589 nm, c=0.10, MeOH).

### N-[3-[(2S,5S)-6-amino-5-fluoro-2,5-bis(fluoromethyl)-2,3,4,5-tetrahydropyridin-2-yl]-4-fluoro-phenyl]-5-methoxy-pyrazine-2-carboxamide

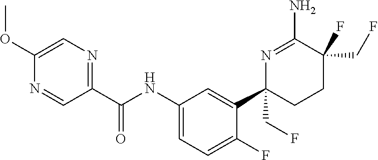 [SMILES: COc1cnc(C(=O)Nc2ccc(F)c([C@]3(CF)CC[C@@](F)(CF)C(N)=N3)c2)cn1]

Prepared from (3R,6S)-6-(5-amino-2-fluorophenyl)-3-fluoro-3,6-bis(fluoromethyl)piperidine-2-thione and 5-methoxypyrazine-2-carboxylic acid ¹H NMR (400 MHz, CDCl₃) δ 9.46 (s, 1H), 8.94 (s, 1H), 8.08 (s, 1H), 7.93-7.89 (m, 1H), 7.50 (dd, J=7.2, 2.8 Hz, 1 Hz), 7.02 (dd, J=11.6, 8.8 Hz, 1H), 4.78-4.74 (m, 1H), 4.68-4.55 (m, 2H), 4.40 (dd, J=47.4, 8.8 Hz, 1H), 4.00 (s, 3H), 2.32-2.27 (m, 1H), 2.17-2.09 (m, 2H), 1.74-1.63 (m, 1H).

### N-[3-[(2S,5R)-6-amino-5-fluoro-2,5-bis(fluoromethyl)-2,3,4,5-tetrahydropyridin-2-yl]-4-fluoro-phenyl]-5-methoxy-pyrazine-2-carboxamide

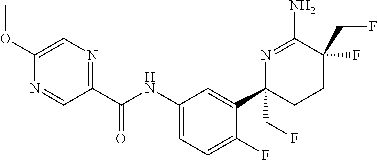 [SMILES: COc1cnc(C(=O)Nc2ccc(F)c([C@]3(CF)CC[C@](F)(CF)C(N)=N3)c2)cn1]

Prepared from (3R,6S)-6-(5-amino-2-fluorophenyl)-3-fluoro-3,6-bis(fluoromethyl)piperidine-2-thione and 5-methoxypyrazine-2-carboxylic acid ¹H NMR (400 MHz, CDCl₃) δ 9.50 (s, 1H), 9.01 (s, 1H), 8.16 (s, 1H), 7.88-7.86 (m, 1H), 7.55 (dd, J=6.8, 2.0 Hz, 1H), 7.11-7.06 (m, 1H), 4.80-4.50 (m, 4H), 4.08 (s, 3H), 2.40-2.34 (m, 1H), 2.27-2.12 (m, 2H), 1.92-1.79 (m, 1H).

### Stereochemistry

The relative stereochemistry of intermediate (3R,6S)-6-(5-amino-2-fluorophenyl)-3-fluoro-3-(fluoromethyl)-6-methylpiperidine-2-thione was assigned by 2D ROESY (rotating frame nuclear Overhauser effect spectroscopy) (Scheme A). nOe (nuclear Overhauser effect) signals were observed between H(A) (δ 4.61) and H(B) (δ 2.10-2.02) and between H(B) (δ 2.10-2.02) and H(D) (δ 1.70) and an nOe signal was also observed between H(C) (δ 4.61) and H(E) (δ 6.58). Thus, the relative stereochemistry of intermediate (3R,6S)-6-(5-amino-2-fluorophenyl)-3-fluoro-3-(fluoromethyl)-6-methylpiperidine-2-thione is confirmed since no significant nOe's was observed between H(A) (δ 4.60) and H(B) (δ 2.53 or 2.13) in the 2D ROESY of (3S,6S)-6-(5-amino-2-fluorophenyl)-3-fluoro-3-(fluoromethyl)-6-methylpiperidine-2-thione. The relative stereochemistries of (3R,6S)-6-(5-amino-2,3-difluorophenyl)-3-fluoro-3-(fluoromethyl)-6-methylpiperidine-2-thione, (3S,6S)-6-(5-amino-2,3-difluorophenyl)-3-fluoro-3-(fluoromethyl)-6-methylpiperidine-2-thione, (3R,6S)-6-(5-amino-2-fluorophenyl)-3-fluoro-3,6-bis(fluoromethyl)piperidine-2-thione and (3S,6S)-6-(5-amino-2-fluorophenyl)-3-fluoro-3,6-bis(fluoromethyl)piperidine-2-thione were assigned by analogy.

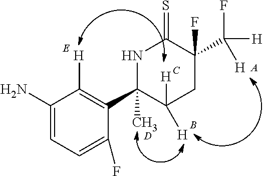

Scheme A. nOe in ROESY of (3R,6S)-6-(5-amino-2-fluorophenyl)-3-fluoro-3-(fluoromethyl)-6-methylpiperidine-2-thione

### Preparation of Compounds of the Invention

### Example 1 N-(3-((2S,5S)-6-amino-5-fluoro-5-(fluoromethyl)-2-methyl-2,3,4,5-tetrahydropyridin-2-yl)-4-fluorophenyl)-5-(methoxy-d₃)pyrazine-2-carboxamide

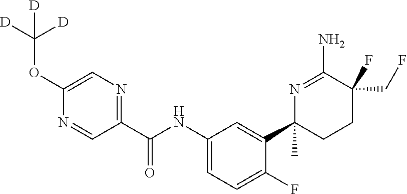 [SMILES: [2H]C([2H])([2H])Oc1cnc(C(=O)Nc2ccc(F)c([C@]3(C)CC[C@@](F)(CF)C(N)=N3)c2)cn1]

Sodium hydride (10 mg, 0.245 mmol, 60% in mineral oil) was added to methanol-d₄ (2 mL). The reaction mixture was stirred for 30 minutes. N-(3-((2S,5S)-6-amino-5-fluoro-5-(fluoromethyl)-2-methyl-2,3,4,5-tetrahydropyridin-2-yl)-4-fluorophenyl)-5-methoxypyrazine-2-carboxamide (20 mg, 0.049 mmol) was added. The reaction mixture was stirred at room temperature for 3 days. The reaction was quenched with saturated aqueous NH₄Cl and water and the organic solvent was removed in vacuo. The mixture was extracted with ethyl acetate. The organic phase was washed with brine, dried over magnesium sulfate, filtered and concentrated. The residue was chromatographed on silica gel to obtain the product. ¹H NMR (600 MHz, DMSO) δ 10.45 (s, 1H), 8.88 (d, J=1.0 Hz, 1H), 8.41 (d, J=1.0 Hz, 1H), 7.83 (dd, J=7.3, 2.4 Hz, 1H), 7.78-7.74 (m, 1H), 7.13 (dd, J=11.8, 8.8 Hz, 1H), 6.12 (brs, 2H), 4.96-4.68 (m, 2H), 2.26-2.04 (m, 2H), 1.96-1.89 (m, 1H), 1.64-1.53 (m, 1H), 1.46 (s, 3H). LC-MS (m/z) 411.4 (MH⁺); t_{R}=0.49 (Method A) The following examples were prepared in a similar manner:

### Example 2 N-[3-[(2S,5R)-6-amino-5-fluoro-5-(fluoromethyl)-2-methyl-2,3,4,5-tetrahydropyridin-2-yl]-4,5-difluoro-phenyl]-5-(methoxy-d₃)pyrazine-2-carboxamide

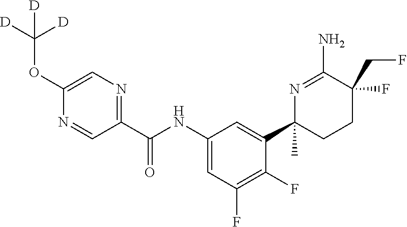 [SMILES: [2H]C([2H])([2H])Oc1cnc(C(=O)Nc2cc(F)c(F)c([C@]3(C)CC[C@](F)(CF)C(N)=N3)c2)cn1]

Prepared from N-[3-[(2S,5R)-6-amino-5-fluoro-5-(fluoromethyl)-2-methyl-2,3,4,5-tetrahydropyridin-2-yl]-4,5-difluoro-phenyl]-5-methoxypyrazine-2-carboxamide. ¹H NMR (600 MHz, DMSO) δ 10.75 (s, 1H), 8.89 (s, 1H), 8.41 (s, 1H), 7.96-7.87 (m, 1H), 7.46 (s, 1H), 6.20 (s, 2H), 4.94 (ddd, J=48.8, 15.3, 11.2 Hz, 1H), 4.55 (ddd, J=45.7, 32.4, 11.1 Hz, 1H), 2.14 (dd, J=8.8, 4.9 Hz, 1H), 2.01 (t, J=17.8 Hz, 1H), 1.78 (t, J=11.8 Hz, 1H), 1.62-1.48 (m, 4H). LC-MS (m/z) 429.1 (MH⁺); t_{R}=1.53 (Method B).

### Example 3 N-[3-[(2S,5R)-6-amino-5-fluoro-2,5-bis(fluoromethyl)-2,3,4,5-tetrahydropyridin-2-yl]-4-fluoro-phenyl]-5-(methoxy-d₃)pyrazine-2-carboxamide

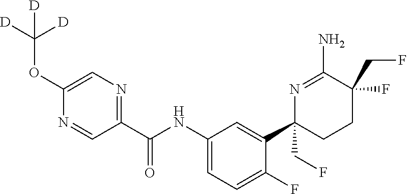 [SMILES: [2H]C([2H])([2H])Oc1cnc(C(=O)Nc2ccc(F)c([C@]3(CF)CC[C@](F)(CF)C(N)=N3)c2)cn1]

Prepared from N-[3-[(2S,5R)-6-amino-5-fluoro-2,5-bis(fluoromethyl)-2,3,4,5-tetrahydropyridin-2-yl]-4-fluoro-phenyl]-5-methoxypyrazine-2-carboxamide. ¹H NMR (600 MHz, DMSO) δ 10.62 (s, 1H), 8.89 (d, J=1.1 Hz, 1H), 8.41 (d, J=1.1 Hz, 1H), 7.86-7.76 (m, 1H), 7.62 (dd, J=7.1, 2.5 Hz, 1H), 7.18 (dd, J=11.8, 8.8 Hz, 1H), 6.38 (s, 2H), 4.96 (ddd, J=48.7, 15.1, 11.1 Hz, 1H), 4.66-4.44 (m, 3H), 2.15-2.08 (m, 1H), 2.08-1.98 (m, 1H), 1.91-1.81 (m, 1H), 1.59-1.43 (m, 1H). LC-MS (m/z) 429.1 (MH⁺); t_{R}=1.42 (Method B).

### Example 4 N-[3-[(2S,5S)-6-amino-5-fluoro-2,5-bis(fluoromethyl)-2,3,4,5-tetrahydropyridin-2-yl]-4-fluoro-phenyl]-5-(methoxy-d₃)pyrazine-2-carboxamide

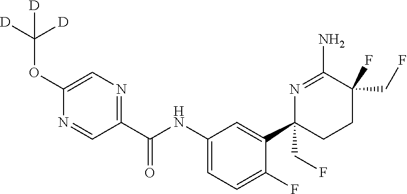 [SMILES: [2H]C([2H])([2H])Oc1cnc(C(=O)Nc2ccc(F)c([C@]3(CF)CC[C@@](F)(CF)C(N)=N3)c2)cn1]

Prepared from N-[3-[(2S,5S)-6-amino-5-fluoro-2,5-bis(fluoromethyl)-2,3,4,5-tetrahydropyridin-2-yl]-4-fluoro-phenyl]-5-methoxypyrazine-2-carboxamide. ¹H NMR (600 MHz, DMSO) δ 10.55 (s, 1H), 8.89 (s, 1H), 8.42 (s, 1H), 7.88 (dd, J=7.3, 2.6 Hz, 1H), 7.84-7.78 (m, 1H), 7.17 (dd, J=11.8, 8.8 Hz, 1H), 6.32 (s, 2H), 4.95-4.64 (m, 2H), 4.52 (td, J=46.8, 8.6 Hz, 2H), 2.20-2.13 (m, 1H), 2.12-2.06 (m, 1H), 1.98 (t, J=13.6 Hz, 1H), 1.56 (q, J=13.6 Hz, 1H). LC-MS (m/z) 429.1 (MH⁺); t_{R}=1.44 (Method B).

### Example 5 N-[3-[(2S,5S)-6-amino-5-fluoro-5-(fluoromethyl)-2-methyl-2,3,4,5-tetrahydropyridin-2-yl]-4,5-difluoro-phenyl]-5-(methoxy-d₃)pyrazine-2-carboxamide

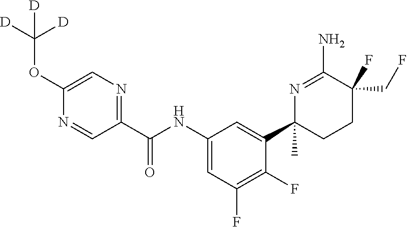 [SMILES: [2H]C([2H])([2H])Oc1cnc(C(=O)Nc2cc(F)c(F)c([C@]3(C)CC[C@@](F)(CF)C(N)=N3)c2)cn1]

Prepared from N-[3-[(2S,5S)-6-amino-5-fluoro-5-(fluoromethyl)-2-methyl-2,3,4,5-tetrahydropyridin-2-yl]-4,5-difluoro-phenyl]-5-methoxypyrazine-2-carboxamide. ¹H NMR (600 MHz, DMSO) δ 10.66 (s, 1H), 8.89 (d, J=0.9 Hz, 1H), 8.42 (d, J=1.0 Hz, 1H), 7.95-7.87 (m, 1H), 7.75-7.68 (m, 1H), 6.10 (s, 2H), 4.96-4.67 (m, 2H), 2.18-2.02 (m, 2H), 1.99-1.87 (m, 1H), 1.67-1.55 (m, 1H), 1.47 (s, 3H). LC-MS (m/z) 429.1 (MH⁺); t_{R}=1.54 (Method B).

### Pharmacological Testing

### Example 6: BACE1 Binding Assay

The binding assay was performed as SPA-based assay using a biotinylated form of human BACE1 recombinantly expressed and subsequently purified from Freestyle HEK293 cells. The binding assay was run in a 50 mM sodium acetate buffer, pH 4.5 containing 50 mM NaCl and 0.03% Tween-20 in white clear bottom 384 plates (Corning #3653). 10 nM (final concentration) radioligand ([³H]-N-((1S,2R)-1-benzyl-3-cyclopropylamino-2-hydroxy-propyl)-5-(methanesulfonyl-methyl-amino)-N-((R)-1-phenyl-ethyl)-isophthalamide) (TRQ11569 purchased from GE Healthcare) was mixed with test compound at a given concentration, 6 nM (final concentration) human BACE1 and 25 μg Streptavidin coated PVT core SPA beads (RPNQ0007, GE Healthcare Life Sciences) in a total volume of 40 Several concentrations of each test compound were tested in the assay for IC₅₀ determination. The plates were incubated for one hour at room temperature and counted in a Wallac Trilux counter. Total and non-specific binding were determined using buffer and 1 μM (final concentration) of the high affinity BACE1 reference inhibitor (S)-6-[3-chloro-5-(5-prop-1-ynyl-pyridin-3-yl)-thiophen-2-yl]-2-imino-3,6-dimethyl-tetrahydro-pyrimidin-4-one, respectively. For each test compound, a IC₅₀ value (the concentration mediating 50% inhibition of the specific binding of the radioligand) was determined from concentration-response curve and used to calculate the Kᵢ from the equation Kᵢ=IC₅₀/(1+L/K_{d}), where L and K_{d} are the final concentration of the radioligand used in the assay and the dissociation constant of the radioligand, respectively. The K_{d} of the radioligand was determined from saturation binding experiments.

TABLE 1
binding affinity of selected compounds

| Compound No | BACE1 Ki (nM) |
|---|---|
| 1 | 17 |
| 2 | 84 |
| 3 | 67 |
| 4 | 14 |
| 5 | 9.8 |

### Example 7: BACE1 Efficacy Assay

The efficacy assay was performed as a FRET-based assay using a commercially available BACE1 kit (Life Technologies, P2985). 2 μl test compound at 10 μM (final concentration) and 15 μl BACE1 enzyme from the kit (final concentration 3 nM) were preincubated for 15 minutes at room temperature before addition of 15 μl of substrate from the kit (250 nM final concentration) and incubated for additional 90 minutes at room temperature. The assay plate was subsequently read in a Pherastar (Ex540/Em590). The enzyme activity observed in presence of test compound were normalized to the enzyme activity observed in presence of buffer and 10 μM (final concentration) of the high affinity BACE1 reference inhibitor (S)-6-[3-Chloro-5-(5-prop-1-ynyl-pyridin-3-yl)-thiophen-2-yl]-2-imino-3,6-dimethyl-tetrahydropyrimidin-4-one, respectively. The efficacy of the test compounds was evaluated at 10 μM (final concentration) and defined as the percent inhibition of the enzyme activity using the equation % inhibition=100%−normalized enzyme activity in percent.

TABLE 2
BACE1 activity of selected compounds

| Compound No | BACE1 inhibition at 10 μM (%) |
|---|---|
| 1 | 100 |
| 2 | 99 |
| 3 | 97 |
| 4 | 97 |
| 5 | 100 |

### MDCK-MDR1 Assay

The permeability of the test compounds was assessed in MDCK-MDR1 cells that were cultured to confluency (4-6 days) in a 96 transwell plate. Test compounds were diluted with the transport buffer (HBSS+1% BSA) to a concentration of 0.5 μM and applied to the apical or basolateral side of the cell monolayer. Permeation of the test compounds from A to B direction or B to A direction was determined in triplicate over a 60-minute incubation time at 37° C. and 5% CO2 with a relative humidity of 95%. Test compounds were quantified by LC-MS/MS analysis based on the peaks area ratios of analyte/IS in both the receiver and donor wells of the transwell plate.

The apparent permeability coefficient Papp (cm/s) was calculated using the equation:

Papp=(dCr/dt)×Vr/(A×CO)

Where dCr/dt is the cumulative concentration of compound in the receiver chamber as a function of time (μM/s); Vr is the solution volume in the receiver chamber (0.05 mL on the apical side; 0.25 mL on the basolateral side); A is the surface area for the transport, i.e. 0.0804 cm² for the area of the monolayer; CO is the initial concentration in the donor chamber (μM).

Compounds are classified Pgp substrates when efflux ratio (Papp BA/Papp AB) is ≥2.

TABLE 3
Efflux ratio of selected compounds

| Compound | MDCK-MDR1 efflux ratio |
|---|---|
| 1 | 1.39 |
| 2 | 0.96 |
| 3 | 0.85 |
| 4 | 0.73 |
| 5 | 0.77 |

As shown in Table 3, the exemplified compounds of the present invention have MDCK-MDR1 efflux ratios below 2 and are thus likely to be able to cross the blood brain barrier (E Kerns, L Di, Drug-like Properties: Concepts, Structure Design and Methods (2008) Elsevier).

### Example 8: Clearance Results

Deuterated compounds were compared to their non-deuterated parent counterparts to determine the impact of deuteration on clearance results

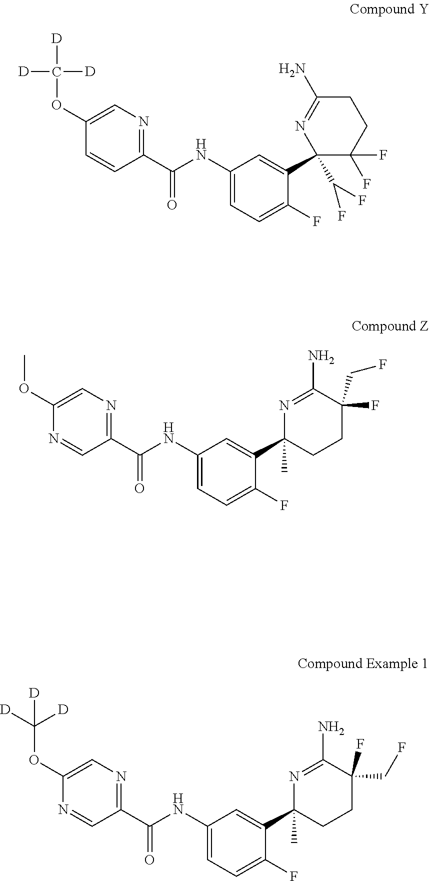 [SMILES: [2H]C([2H])([2H])Oc1ccc(C(=O)Nc2ccc(F)c([C@@]3(C(F)F)N=C(N)CCC3(F)F)c2)nc1 ; COc1cnc(C(=O)Nc2ccc(F)c([C@]3(C)CC[C@@](F)(CF)C(N)=N3)c2)cn1 ; [2H]C([2H])([2H])Oc1cnc(C(=O)Nc2ccc(F)c([C@]3(C)CC[C@@](F)(CF)C(N)=N3)c2)cn1]

TABLE 4

| | | | CLᵢₙₜ, | CLᵢₙₜ, | CLᵢₙₜ, | |
|---|---|---|---|---|---|---|
| | | | Human | Rat | Dog | CLᵢₙₜ, Cyno |
| | Concentration | | (L/h/kg) ± | (L/h/kg) ± | (L/h/kg) ± | (L/h/kg) ± |
| Compound | (microM) | | SD | SD | SD | SD |
| Compound Y | 0.1 | Microsomes | — | — | — | — |
| | | Hepatocytes | <0.1 | 1.4 ± 0.9 | 98 ± 6 | 80 ± 10 |
| | 0.3 | Microsomes | — | — | — | — |
| | | Hepatocytes | <0.1 | 1.0 ± 0.7 | 84 ± 5 | 32 ± 9 |
| | 1 | Microsomes | 0.5 ± 0.1 | 1.0 ± 0.2 | 21 ± 2 | 12 ± 4 |
| | | Hepatocytes | 0.29 ± 0.2 | 2.1 ± 0.6 | 57 ± 5 | 14 ± 1 |
| Compound Z | 0.1 | Microsomes | 1.0 ± 0.34 | 2.7 ± 0.7 | 40 ± 7 | 70 ± 15 |
| | | Hepatocytes | 1.0 ± 0.29 | 1.7 ± 1.4 | 112 ± 34 | 137 ± 8 |
| | 0.3 | Microsomes | 0.75 ± 0.18 | 1.8 ± 0.5 | 31 ± 2 | 61 ± 7 |
| | | Hepatocytes | 0.95 ± 0.16 | 1.5 ± 0.7 | 101 ± 26* | 135 ± 31 |
| | 1 | Microsomes | 0.51 ± 0.01 | 1.3 ± 0.1 | 25 ± 2 | 34 ± 1 |
| | | Hepatocytes | 0.72 ± 0.36 | 1.0 ± 0.9 | 64 ± 14 | 75 ± 21 |
| Compound | 0.1 | Microsomes | — | — | — | — |
| Example 1 | | Hepatocytes | <0.1 | 1.5 ± 0.9 | 34 ± 2 | 75 ± 10 |
| | 0.3 | Microsomes | — | — | — | — |
| | | Hepatocytes | <0.1 | 1.2 ± 0.8 | 25 ± 3 | 74 ± 4 |
| | | Microsomes | 0.20 ± 0.04 | 0.9 ± 0.4 | 5 ± 1 | 10 ± 4 |
| | 1 | Hepatocytes | <0.1 | <0.4 | 11 ± 2 | 20 ± 6 |

An overview of clearance results of related compounds in microsomes and hepatocytes in 4 different species

### Example 8: In Vivo Pharmacological Testing

Assessment of AR levels in rat brain and plasma following BACE1 inhibition.

### Animals.

All rodent care and experimental procedures were approved by Lundbeck Veterinary Staff, according to Danish legislature. The rodents were maintained in a barrier facility with a 12/12-h light/dark cycle and ad libitum food and water access.

Treatment of Naïve Rats.

Young adult Male Sprague Dawley rats of approximately 250 g weight were purchased from Charles River and received vehicle (2.5% HP betaCD+1M MeSO₄, pH 2.5) or 0-40 mg/kg of test compounds (dissolved in vehicle) by oral gavage (p.o). The compounds are dosed at a volume of 5 ml/kg. Cohorts of 5-10 animals were established for each treatment condition.

The animals undergoing treatment were closely monitored by veterinary staff for any signs of toxicity. Monitoring parameters included body weight, physical appearance, changes in coat appearance, occurrence of unprovoked behavior, and blunted or exaggerated responses to external stimuli.

### Tissue Collection.

Studies were set up as acute dose response treatment studies were samples were taken at T=180 minutes after initial dosing, or as 12 h or 24 h timecourse treatment studies. At the end of the test period, the animals were stunned and decapitated with a guillotine. Trunk-blood was sampled in EDTA coated tubes after decapitation of the animal. The blood was centrifuged at 2200G at 4° C. for 15 minutes and the plasma was collected and frozen at −80° C. The blood was aliquoted for Aβ ELISA and DMPK analysis. Immediately following sacrifice, the brain was extracted and split into 2 halves. The left half was dissected; with the front forebrain taken for Aβ ELISA and the remainder used for DMPK analysis. These samples were also snap frozen on dry ice and stored at −80° C. until use for analysis. The right hemibrains were snap frozen on dry ice and stored at −80° C. for independent confirmatory Aβ ELISA evalution.

### Tissue Processing.

The cortex samples were thawed slightly on wet ice before they were homogenized with a small volume dispersing instrument (T10 basic ULTRA-TURRAX®) which was set at speed 5 for approximately 5-7 sec. The tissue was processed in a 10 times volume of the weight, for example 100 mg of tissue was homogenized in 10004 of Homogenization buffer. Homogenization buffer: 50 ml Milli Q water+50 nM NaCl+0.2% Diethylamin (DEA)+1 tablet of Complete Protease inhibitor cocktail+1 nM 4-(2-aminoethyl) benzenesulfonyl fluoride hydrochloride irreversible serine protease inhibitor (AEBSF).

After homogenization 450 μL aliquots of the samples are collected into a 1.5 ml Eppendorf tube and placed on wet ice, 0.5% NP-40 (50 ul) was added to all samples and then they were incubated on ice for 30 min. After which all samples were sonicated using an Ultrasonic homogenizer with 20 kHz homogeneous sound (SONOPLUS HD2070, Bandelin Electronic) 10 pulse set at 12-13% power to extract all the Aβ species. The samples were then centrifuged (Ole Dich 157 MPRF Micro centrifuge) at 20000G for 20 minutes at 4° C. After centrifugation 2854 of the supernatant was pipetted into 600 μL microtubes tubes and neutralized with 154 of 1M Tris-HCL buffer.

### ELISA Protocol.

WAKO 294-62501 Human/Rat Abeta amyloid-40 kit was used for all ELISA analyses. 30 μL plasma samples or 30 μL of the cortex supernatants generated as described above were placed in 600 μL microtubes tubes on wet ice. To this 30 μL of 8M Urea (AppliChem A1049, 9025) are added to generate a 2-fold dilution. Both plasma and cortex supernatants are incubated on ice for 30 min. Standard rows were prepared from the standard peptide stock provided in the kit and standard diluent containing 1.6M Urea (200 μL 8M Urea+800 μL of standard diluent) and 0.8M Urea (400 μL 8M Urea+36004 Standard diluent). A serial 2-fold dilution of Aβ40 from 100 pmol/ml to 0 pmol/L was prepared for the assay.

After incubation with urea, all samples were further diluted by addition of 5 times standard diluent from the Kit. This was done by adding 240 μL Standard Diluent to 60 μL sample/urea mixture, which was then mixed well. 100 μL of each diluted sample was pipetted into designated wells of the ELISA plate in duplicates. The plate was then covered and incubated overnight at 4° C. The following day, the ELISA kit was brought to room temperature before use. The incubated plate was washed 5 times with the 20× washing solution diluted in Milli Q water. 100 μL HRP-conjugate was applied to each well, and the plate was covered and incubates at 4° C. for 1 hr. The wash was repeated again for 5 times. 100 μL 3,3′,5,5′-Tetramethylbenzidine (TMB) solution was applied to each well and the plate was covered and incubated in the dark at room temperature for 30 minutes. 100 μL STOP-solution was next applied to each well, and the plate was read at 450 nm wavelength in a spectrophotometer (Labsystems Multiscan Ascent) within 30 min of adding the STOP-solution to the wells.

Concentration of AR in the samples was determined based on a standard curve generated from standards containing known concentrations of synthetic Aβ40. Those skilled in the art will appreciate that diethylamine (DEA) and urea extractions will release soluble Aβ, and insoluble Aβ respectively. Since the ELISA kit is validated and widely used, it is accepted that as long as the treatment conditions and assay conditions are the same for each compound tested, then the assay should yield consistent robust data for the compounds tested and produce minimal discrepancies.

### Data Analysis

To determine the concentration of Aβ40 in the samples, the interpolated values of the samples loaded on plates are multiplied by 20 to account for the dilutions made when the volumes of DEA, urea and neutralization solution were added up. Values are calculated as percentage change in Aβ40 compared to vehicle treated animals.

### Bioanalysis of Brain and Plasma Samples

TC was determined in plasma and brain homogenate using UltraPerformance LC® (UPLC®) chromatography followed by tandem-MS (MS/MS) detection.

Apparatus:

Tecan Genesis RSP 200; Biomek NXP, Beckman Coulter; Sigma 4K15 centrifuge; Acquity UPLC, Waters; Sciex AP14000 TQ, Applied Biosystems; MS software: Analyst version 1.4.1

### Chemicals

Acetonitrile, HPLC-grade, Fluka, No. 34967N; Methanol, HPLC-grade, Sigma-Aldrich, Lot 9003S; Formic acid, HPLC-grade, Riedel-de Haën, Lot 51660; Purified water, Millipore Synergy UV

### Sample Preparation

Brain homogenate was prepared by homogenizing the brain 1:4 (v/v) with water:2-propanol:DMSO (50:30:20 v/v/v) followed by centrifugation and collection of the supernatant. Calibration standards and QC samples were prepared using a Hamilton robot. 150 μL of ISTD in acetonitrile (1 ng/mL ISTD) was added to 25 μL of calibration standards, QC samples and test samples (plasma and brain homogenate) using a Biomek robot. After centrifugation (6200 g, 4° C., 20 min) 100 μL supernatant from each sample was transferred to a new plate and mixed with 100 μL water with 0.1% formic acid using a Biomek robot (method file InVivo transfer). After a quick centrifugation (6200 g, 4° C., 5 min) the samples were placed in the auto-sampler.

### UPLC-MS/MS Analysis

MS/MS detection was done with an Applied Biosystems Sciex API 4000 instrument in positive-ion electrospray ionisation mode. TC and ISTD were detected at a parent>daughter mass to charge ratio (m/z). Nitrogen was used for the nebulizer and collision gases. The peak area correlated linearly with the plasma and brain concentration of the analytes in the range of 1.00-1000 ng/mL plasma and 5.00-5000 ng/g brain (corrected for dilution). If the plasma/brain sample drug concentration was above 1000 ng/mL or 5000 ng/g, the sample was diluted appropriately in blank plasma/blank brain homogenate before analysis.

### Chromatographic System

Analytical columns: Waters Acquity UPLC HSS C18 SB (pH 2-8) 1.8 μm, 2.1×30 mm.

Mobile phase A: 0.1% aq. formic acid or 0.1% aq. ammonium hydroxide

Mobile phase B: Acetonitrile with 0.1% aq. formic acid or 0.1% aq. ammonium hydroxide.

Weak wash: Methanol

Strong wash: Acetonitrile/Isopropanol/formic acid (50/50/2 v/v/v)

Flow: 0.6 mL/min

Run time: 3 min.

To waste: 0-0.5 min

Temperature: 40° C.

### Gradient:

| Time (min) | % A | % B |
|---|---|---|
| 0 | 98 | 2 |
| 0.01 | 98 | 2 |
| 1.5 | 5 | 95 |
| 2 | 5 | 95 |
| 2.2 | 98 | 2 |
| 3 | 98 | 2 |

TABLE 1
Results for compound Example 1

| | | | Mean | | | |
|---|---|---|---|---|---|---|
| | | Mean rat | rat | | | Aβ40 |
| | | brain | plasma | Brain/ | Aβ40 | reduction |
| Dose | | conc. | conc. | Plasma | reduction | (%) |
| (mg/kg) | Time (h) | (ng/g) | (ng/ml) | ratio | (%) brain | plasma |
| 10 | 12 | 140 | 79 | 1.7 | 13.7 | 20.7 |
| | 6 | 430 | 230 | 1.9 | 39.4 | 15.5 |
| | 3 | 700 | 340 | 2.1 | 38.3 | 21.7 |
| | 1 | 1100 | 500 | 2.3 | 17.2 | 29.5 |
| | 0.5 | 550 | 300 | 1.8 | −0.3 | −0.4 |

TABLE 2
Results for compound Example 1

| | | | Mean | | | |
|---|---|---|---|---|---|---|
| | | Mean rat | rat | | | Aβ40 |
| | | brain | plasma | Brain/ | Aβ40 | reduction |
| Dose | | conc. | conc. | Plasma | reduction | (%) |
| (mg/kg) | Time (h) | (ng/g) | (ng/ml) | ratio | (%) brain | plasma |
| 20 | 12 | 437.5 | 158.8 | 2.7 | 34.5 | 41.3 |
| | 6 | 1540.9 | 477.1 | 3.2 | 42.6 | 36.9 |
| | 3 | 1961.2 | 571.9 | 3.4 | 38.5 | 34.5 |
| | 1 | 2383.9 | 613.9 | 3.9 | 29.7 | 41.4 |
| | 0.5 | 1531.4 | 532.2 | 2.9 | 14.7 | 36.5 |

TABLE 3
Results for compound Example 1

| | | | Mean | | | |
|---|---|---|---|---|---|---|
| | | Mean rat | rat | | | Aβ40 |
| | | brain | plasma | Brain/ | Aβ40 | reduction |
| Dose | | conc. | conc. | Plasma | reduction | (%) |
| (mg/kg) | Time (h) | (ng/g) | (ng/ml) | ratio | (%) brain | plasma |
| 40 | 12 | 1100.0 | 410.0 | 2.5 | 57.7 | 41.5 |
| | 6 | 3700.0 | 1200.0 | 3 | 59.5 | 49.9 |
| | 3 | 3000.0 | 1100.0 | 2.8 | 50.6 | 47.1 |
| | 1 | 2800.0 | 880.0 | 3.1 | 43.9 | 47.8 |
| | 0.5 | 2500.0 | 740.0 | 2.8 | 39.8 | 43.0 |

As shown in tables 1, 2, and 3 compounds in the present invention are able to penetrate the blood brain barrier and show significant efficacy in the CNS.

1. A compound of Formula I 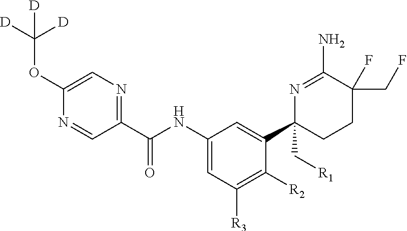
wherein R₁ is hydrogen or fluorine;
R₂ is hydrogen or a halogen;
R₃ is hydrogen or a halogen; and
D is deuterium,
or a pharmaceutically acceptable salt thereof.

2. The compound according to claim 1, or pharmaceutically acceptable salt thereof, wherein formula I is formula Ia or Ib 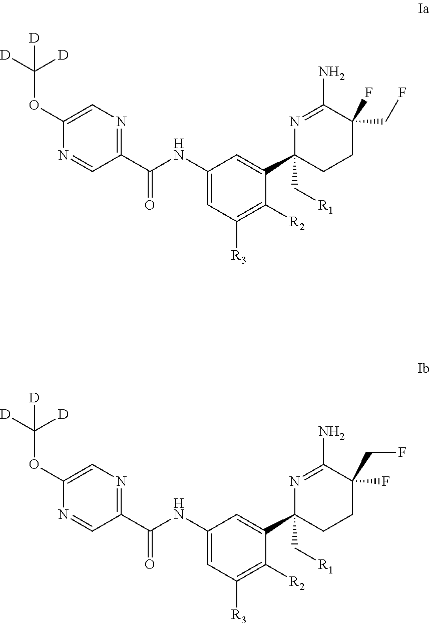
wherein R₁ is hydrogen or fluorine;
R₂ is hydrogen or a halogen; and
R₃ is hydrogen or a halogen.

3. The compound according to claim 1, or pharmaceutically acceptable salt thereof, wherein each of R₁, R₂, and R₃ is hydrogen.

4. The compound according to claim 1, or pharmaceutically acceptable salt thereof, wherein at least one of R₂ and R₃ is a halogen.

5. The compound according to claim 1, or pharmaceutically acceptable salt thereof, wherein at least one of R₁, R₂ and R₃ is fluorine.

6. The compound according to claim 1, or pharmaceutically acceptable salt thereof, wherein at least R₂, is a halogen.

7. The compound according claim 1, or pharmaceutically acceptable salt thereof, wherein at least R₂ is fluorine.

8. A compound according to claim 1 selected from the group consisting of
N-(3-((2S,5S)-6-amino-5-fluoro-5-(fluoromethyl)-2-methyl-2,3,4,5-tetrahydropyridin-2-yl)-4-fluorophenyl)-5-(methoxy-d₃)pyrazine-2-carboxamide;
N-[3-[(2S,5R)-6-amino-5-fluoro-5-(fluoromethyl)-2-methyl-2,3,4,5-tetrahydropyridin-2-yl]-4,5-difluoro-phenyl]-5-(methoxy-d₃)pyrazine-2-carboxamide;
N-[3-[(2S,5R)-6-amino-5-fluoro-2,5-bis(fluoromethyl)-2,3,4,5-tetrahydropyridin-2-yl]-4-fluoro-phenyl]-5-(methoxy-d₃)pyrazine-2-carboxamide;
N-[3-[(2S,5S)-6-amino-5-fluoro-2,5-bis(fluoromethyl)-2,3,4,5-tetrahydropyridin-2-yl]-4-fluoro-phenyl]-5-(methoxy-d₃)pyrazine-2-carboxamide; and
N-[3-[(2S,5S)-6-amino-5-fluoro-5-(fluoromethyl)-2-methyl-2,3,4,5-tetrahydropyridin-2-yl]-4,5-difluoro-phenyl]-5-(methoxy-d₃)pyrazine-2-carboxamide,
or pharmaceutically acceptable salts thereof.

9. A method of treating a neurodegenerative or cognitive disorder comprising administering a therapeutically effective amount of a compound according to claim 1, or a pharmaceutically acceptable salt thereof, to a patient in need thereof.

10. A pharmaceutical composition comprising a compound according to claim 1, or a pharmaceutically acceptable salt thereof and a pharmaceutically acceptable carrier.

11. A method of treating a disease selected from the group consisting of Alzheimer's disease, preclinical Alzheimer's disease, prodromal Alzheimer's disease, mild cognitive impairment, Down's syndrome and cerebral amyloid angiopathy, the method comprising administering a therapeutically effective amount of a compound according to claim 1, or a pharmaceutically acceptable salt thereof, to a patient in need thereof.

12. (canceled)

13. (canceled)

14. A pharmaceutical composition comprising:
a compound of Formula I according to claim 1, or a pharmaceutically acceptable salt thereof; and
a second pharmaceutical compound, wherein said second pharmaceutical compound is effective, alone or in combination with the compound of Formula I or a pharmaceutically acceptable salt thereof.

15. A method for the treatment of a disease selected from the group consisting of Alzheimer's disease, preclinical Alzheimer's disease, prodromal Alzheimer's disease, mild cognitive impairment, Down's syndrome and cerebral amyloid angiopathy comprising administering a combination of a compound of Formula I, or a pharmaceutically acceptable salt thereof, to a patient in need thereof, with a second pharmaceutical compound, wherein said second pharmaceutical compound is effective, alone or in combination with the compound of Formula I or a pharmaceutically acceptable salt thereof.

16. The method according to claim 15, wherein the combination may be by means of a co-formulation, by means of separate formulations and simultaneous administration, or by means of separate formulations and non-simultaneous administration as part of an overall treatment regime.

17. The compound according to claim 2, or pharmaceutically acceptable salt thereof, wherein each of R₁, R₂, and R₃ is hydrogen.

18. The compound according to claim 2, or pharmaceutically acceptable salt thereof, wherein at least one of R₂ and R₃ is a halogen.

19. The compound according to claim 2, or pharmaceutically acceptable salt thereof, wherein at least one of R₁, R₂, and R₃ is fluorine.

20. The compound according to claim 2, or pharmaceutically acceptable salt thereof, wherein at least R₂ is a halogen.

21. The compound according claim 2, or pharmaceutically acceptable salt thereof, wherein at least R₂ is fluorine.

22. A pharmaceutical composition comprising a compound according to claim 8, or a pharmaceutically acceptable salt thereof; and a pharmaceutically acceptable carrier.
